SEQUENCE LISTING (9 sequences)

SEQ ID NO: 1 - DNA, 78 bp, Staphylococcus epidermidis
ttattttttgaatttattaactgtatcgataatccattttactaaatcaccgattgtagaaatgatatctgctgccat

SEQ ID NO: 2 - protein, 25 aa, Staphylococcus epidermidis
MAADIISTIGDLVKWIIDTVNKFKK

SEQ ID NO: 3 - protein, 25 aa, Staphylococcus epidermidis
MAQDIISTIGDLVKWIIDTVNKFKK

SEQ ID NO: 4 - protein, 25 aa, Staphylococcus epidermidis
MAADIISTIGDLVKWIIDTVNKFTK

SEQ ID NO: 5 - protein, 25 aa, Staphylococcus epidermidis
MAQDIISTIGDLVKWIIDTVNKFTK

SEQ ID NO: 6 - protein, 25 aa, Staphylococcus epidermidis
MAQDIISTISDLVKWIIDTVNKFTK

SEQ ID NO: 7 - protein, 23 aa, Staphylococcus epidermidis
MSIVSTIIEVVKTIVDIVKKFKK

SEQ ID NO: 8 - protein, 5 aa, Artificial Peptide
  Artificial peptide linker
GGGGS

SEQ ID NO: 9 - protein, 22 aa, Artificial Peptide
  Artificial Peptide Linker
GGGGGGSMFGGAKKRSGGGGGG

(19) United States
(12) Patent Application Publication
(10) Pub. No.: US 2010/0166708 A1
(43) Pub. Date: Jul. 1, 2010

(54) ANTIMICROBIAL AND ANTI-INFLAMMATORY THERAPIES AND COMPOSITIONS

(75) Inventors: Richard L. Gallo, San Diego, CA (US); Victor Nizet, San Diego, CA (US); Anna Cogen, Silverdale, WA (US); YuPing Lai, San Diego, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, Oakland, CA (US)

(21) Appl. No.: 12/528,074

(22) PCT Filed: Feb. 20, 2008
(86) PCT No.: PCT/US08/54453

§ 371 Date: Mar. 5, 2010

Related U.S. Application Data
(60) Provisional application No. 60/890,683, filed on Feb. 20, 2007.

(51) Int. Cl. A61K 38/16; A01N 37/18; A61K 35/00; A61K 35/74; A61K 38/12; A01P 1/00; A61P 31/04; A61P 17/00; A61P 31/10; A61P 33/00; A61P 31/12; A61P 17/02; A61P 29/00

(52) U.S. Cl. 424/93.2; 514/12; 514/11; 424/93.42; 424/780

(57) ABSTRACT

The disclosure provides methods and compositions useful for treating microbial and viral infections. In certain aspects, the compositions and methods relate to the use of an effective amount of a delta-haemolysin and/or phenol soluble modulin-delta or functional variant thereof. In other aspects, the compositions and methods relate to the use of an effective amount of Staphylococcus epidermidis or an extract of 5. epidermidis comprising delta-haemolysin and/or phenol soluble modulin-delta or functional variant thereof.

### CROSS REFERENCE TO RELATED APPLICATIONS

This application claims priority under 35 U.S.C. §119 from Provisional Application Ser. No. 60/890,683, filed Feb. 20, 2007, the disclosure of which is incorporated herein by reference.

### STATEMENT AS TO FEDERALLY SPONSORED RESEARCH

The U.S. Government has certain rights in this disclosure pursuant to Grant No. AI052453 awarded by the National Institutes of Health.

### TECHNICAL FIELD

The disclosure relates to peptides, biological agents, cell preparations and cell-free preparations and methods to treat viral and other microbial infections and to treat inflammatory diseases and disorders.

### BACKGROUND

Virus infections occur following entrance of virions into host cells by a variety of mechanisms including endocytosis of non-enveloped viruses and fusion with the cell membrane by enveloped viruses. One primary barrier to the infection is epithelial keratinocyte of the skin. Alterations in skin barrier function are seen in atopic dermatitis (AD). This finding may contribute to infection with bacteria and selected viruses, including Herpesviridae (herpes simplex virus (HSV), varicella-zoster virus) and vaccinia virus. However, it is unlikely that a defect in the physical barrier alone accounts for the remarkably increased susceptibility of AD patients to recurrent skin infections. Patients with plaque psoriasis, a common Th1-mediated inflammatory skin disease also associated with skin barrier dysfunction, do not have increased susceptibility to microbial skin infection. Additionally, inflammation is associated with viral infection, bacterial infection, contact with other inflammatory agents and/or autoimmune diseases or disorders.

Inflammation is a key element of the innate immune system in the response to a variety of challenges, including those caused by bacterial and viral infections as well as by damaged or dying host cells. It is well understood that resolution of inflammation is essential for maintaining the balance between health and disease. Excessive uncontrolled inflammation results in a variety of pathological conditions and evolution of the inflammatory responses is thus a result of a trade-off between its beneficial and detrimental effects.

### SUMMARY

Provided are compositions and methods useful to control and eradicate infection using the peptides delta-haemolysin and phenol soluble modulin-delta. Such peptides can be purified or produced by Staphylococcus epidermidis (S. epidermidis). S. epidermidis is a common microflora on the skin. S. epidermidis has been found to inhibit the growth of Staphylococcus aureus and Group A Streptococcus, the two leading causes of human skin infections and wound infections. Using biochemical methods various agents were identified in S. epidermidis that inhibit microbial infection. In one embodiment, the agents are delta-haemolysin and phenol soluble modulin-delta. These peptides have both bacteriostatic and bactericidal activity. S. epidermidis, delta-haemolysin and phenol soluble modulin-delta may be used in the treatment of infectious skin disorders.

Delta-haemolysin or phenol soluble modulin-delta alone or in combination, or an extract of S. epidermidis can be applied topically or administered systemically to reduce the severity of infection caused by microbial infection (including S. aureus and Group A Streptococcus). This disclosure makes use of the normal bacteria that live on the skin to protect against disease causing bacteria. Because of its natural abundance, it is predicted to be effective, and inexpensive to make. Treatment of infectious skin disorders with delta-haemolysin, phenol soluble modulin-delta or S. epidermidis would result in clearance of infection caused by Group A Streptococcus or S. aureus.

The disclosure provides a novel anti-infective treatment to be compared with existing antibiotics or surface antiseptics. Treatment of skin or systemic disorders, infectious or non-infectious, with topical application of delta-haemolysin, phenol soluble modulin-delta or a functional variant thereof would result in faster recovery from many dermatological diseases, including wounds, diabetic ulcers, acne, rosacea, atopic dermatitis, pyodermas, burn wounds, catheter infections, Group A Streptococcus, Staphylococcus aureus and other dermatological diseases. A combination product of cathelicidin and delta-haemolysin and/or phenol soluble modulin-delta can be created for an antimicrobial therapy or to enhance the immune response against tumors or to accelerate wound healing. Delta-haemolysin and/or phenol soluble modulin-delta plus cathelicidin can be administered systemically to treat systemic infections, in particular Group A Streptococcus and S. aureus (including MRSA) in normal and immunocompromised patients. Delta-haemolysin and/or phenol soluble modulin-delta could be used alone or in combination with other agents, such as cathelicidin, to create a combination product for an antimicrobial therapy or to accelerate wound healing.

The disclosure demonstrates that delta-haemolysin and phenol soluble modulin-delta have both bacteriostatic and bactericidal activity. Accordingly, S. epidermidis (or a genetically engineered organism that expresses delta haemolysin and/or phenol soluble modulin-delta) and delta-haemolysin and/or phenol soluble modulin-delta may be used in the treatment of infectious skin disorders.

The disclosure provides a method for inhibiting the growth of a bacterium or yeast comprising contacting the bacterium or yeast with an inhibiting effective amount of a composition comprising a delta haemolysin and/or a phenol soluble modulin-delta.

The disclosure also provides a method of treating infections or dermatological disorders comprising administering an effective amount of Staphylococcus epidermidis (S. epidermidis), or an effective amount of an extract of S. epidermidis comprising the peptide delta-haemolysin and/or phenol soluble modulin-delta.

The disclosure also provides a composition comprising (i) S. epidermidis, (ii) a recombinant host cell that expresses a peptide comprising delta haemolysin and/or phenol soluble modulin-delta or functional variant thereof; (iii) an extract of (i) or (ii) comprising a delta haemolysin and/or phenol soluble modulin-delta or functional variant thereof; and (iv) delta haemolysin and/or phenol soluble modulin-delta or a functional variant thereof.

In one aspect, a method is provided for treating or preventing an inflammatory or autoimmune disease or disorder. The method comprises administering to a subject in need thereof an effective amount of Staphylococcus epidermidis or S. epidermidis 10 ka filtrate. In another aspect, a method is provided for treating or preventing a skin infection. The method comprises administering to a subject in need thereof an effective amount of Staphylococcus epidermidis or S. epidermidis 10 ka filtrate.

The disclosure further provides compositions comprising an LTA agent. The LTA agent can be derived from a Staphylococcus sp. or a fraction thereof (e.g., a 10 kDa fraction). An LTA agent is useful in the treatment of an inflammatory diseases and disorders. The LTA agent can be used in methods to treat skin inflammatory diseases and disorder.

The disclosure also provides a composition comprising (i) a recombinant host cell that expresses an LTA comprising or (ii) an extract of (i) comprising a LTA.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-B shows S. epidermidis inhibits growth of Group A Streptococcus (GAS). (a) S. epidermidis was added to a GAS lawn on a THA plate. The clear zones around the S. epidermidis colony indicates inhibition of GAS growth. (b) Growth of GAS in 50% stationary phase S. epidermidis supernatants, was measured by turbidity (OD₆₀₀). As a control, GAS was grown in 50% stationary phase GAS supernatants. Thus, lack of growth of GAS does not result from nutrient depletion but presence of inhibitory agent produced by S. epidermidis.

FIG. 2A-C shows partial purification of S. epidermidis bacteriocin. (a) S. epidermidis supernatants purified over a C18 reversed phase Sep Pak column. Activity is seen in flow through and 80% acetonitrile/0.1% TFA elution fractions. (b) HPLC of 80% elution from part (a) over C18 column. (c) radial diffusion assay shows anti-GAS activity in fraction 37, 72% ACN.

FIG. 3 shows MS-TOF data.

FIG. 4 shows exemplary data regarding minimal inhibitory and killing activity of a synthetic peptide of the disclosure.

FIG. 5 shows a helical wheel of amino acid characteristics as it relates to delta haemolysin of SEQ ID NO:2.

FIG. 6A-F shows Staphylococcus epidermidis inhibits the growth of Group A Streptococcus in vitro and in vivo. In vitro, S. epidermidis inhibits growth and survival of GAS as evidenced by (a) zone of GAS inhibition surrounding S. epidermidis, ATCC 12228 colony, (b) GAS growth suppression in S. epidermidis, ATCC 12228, supernatants, (c) GAS killing by partially purified S. epidermidis, ATCC 12228, 1457, and RP62A supernatants and radial diffusion growth inhibition (d) in partially purified S. epidermidis, ATCC 12228, supernatants. In vivo assays illustrate that S. epidermidis inhibits GAS growth on the skin. (e) delta-hemolysis by GAS shows that S. epidermidis, but not Lactococcus lactis (LL) prevents survival of GAS on finger tips. (f) GAS survival on mouse skin prepopulated with PBS, S. epidermidis 1457 or 12228.

FIG. 7A-B shows S. epidermidis prevents growth of S. aureus, in vitro. S. epidermidis, ATCC 12228, 1457, and RP62A supernatants were concentrated using reversed-phase C18 SepPak column, and components eluted with 80% acetonitrile/0.1% trifluoroacetic acid, lyophilized, and resuspended in water. (a) Radial diffusion assay, with GAS NZ131, show inhibitory zones. (b) GAS growth curves shown GAS killing by all strains of S. epidermidis.

FIG. 8A-F shows identification and purification of delta-haemolysin and phenol soluble modulin-delta from S. epidermidis supernatant. S. epidermidis, ATCC 12228, supernatant was purified using SepPak C18 column. (a) Fraction eluted with 80% acetonitrile/0.1% TFA was further fractionated using a C₁₈ column and HPLC. (b) Fraction 37 inhibits GAS in radial diffusion assay. (c) and (d) MS TOF-TOF sequence results identify the peptides delta-haemolysin and phenol soluble modulin-delta in fraction 37. (e) Minimal inhibitory concentrations (MIC) and minimal bactericidal concentrations (MBC) using synthetic peptides. (f) GAS MIC of delta-haemolysin with and without 2 μM PSM-delta.

FIG. 9A-B shows allelic exchange mutagenesis of PSMdelta. (a) Allelic exchange mutagenesis scheme to replace psmdelta with cat, to create S. epidermidis deltapsmdelta. (b) Radial diffusion assay (RDA) of GAS with supernatant from S. epidermidis 1457 WT and S. epidermidis deltapsmdelta shows larger inhibitory area from WT. Average area of RDA, p=0.1, n=2, Unpaired Students T-test.

FIG. 10A-F shows PSM-delta and delta-haemolysin interact with membranes and cause mechanical disruption. (a) Tryptophan emission signal increases intensity with increasing concentration of delta-haemolysin. (b) Unfolding curves of delta-haemolysin in the presence of urea, showing dissociation of complexes. (c) Table of slope (m), midpoint (Dₘ), and deltaG¹(H₂O) shows delta-haemolysin interaction with subsequent stabilization of multimers. 25 μM delta-haemolysin increases midpoint of unfolding curve to 3.2 M urea, and deltaG¹(H₂O)=1.57 kCal*mol⁻¹. (d-e) POPC/POPG vesicles encapsulating ANTS/DPX show leakage in the presence of increasing concentrations of delta-haemolysin and PSM-delta. (f) Membrane disruption observed in SEM analysis of GAS membranes after incubation with 1×PBS, 16 μM CRAMP, and 16 μM delta-haemolysin.

FIG. 11A-D shows delta-haemolysin interacts strongly with lipid membranes. Sequestration of tryptophan (Trp) can be observed through blue shift in wavelength of photon emission after excitation at 290 nm. (a) In the presence of vesicles, delta-haemolysin emission blue shifts indicating that the tryptophan is more shielded from the aqueous environment. This is also true in the presence of urea (b). The strong association of the peptide with lipid membranes is evidenced by urea's inability to dissociate delta-haemolysin from the vesicle and delta-haemolysin strongly blue-shifted state. (c) overlay of emission spectra. (d) Table of shifts in maximum wavelengths in the presence of vesicles and/or urea.

FIG. 12A-C shows peptide-lipid and peptide-peptide interactions affect secondary structures of delta-haemolysin and PSM-delta. Circular dichroism spectra of (a) 20 μM delta-haemolysin or (b) 20 μM PSM-delta in the presence and absence of POPC/POPG vesicles. (c) Spectra of 20 μM delta-haemolysin in the presence of increasing concentrations of PSM-delta. Spectra of PSM-delta spectra showed minimal secondary structure.

FIG. 13A-G shows Staphylococcus epidermidis suppresses the production of TLR3-dependent TNFα. (a) TLR3-dependent TNFα suppressed by S. epidermidis 10 KDa filtrate. (b) and (c) S. epidermidis 10 KDa filtrate suppressed TLR3-dependent TNFα on mRNA and protein levels in time-dependent manner. (d) TLR3-dependent TNFα is specifically suppressed by 10 KDa filtrates from three S. epidermidis strains and one S. aureus strain. (e) and (f) S. epidermidis 10 KDa filtrate inhibited interleukin-8 and interleukin-6 induced by TLR3 ligand. (g) S. epidermidis 10 KDa filtrate had no effect on TLR3-induced interferon β. n.s., no significance. Lens magnification was 60×. In (a), (e) and (f), ***P<0.001. P-values were determined by Two-tailed t tests (GraphPad Prism4). In (b) and (c), **P<0.01 and ***P<0.001. P-values were evaluated by GraphPad Prism4 Two-way ANOVA. d, *P<0.05, **P<0.01 and ***P<0.001. P-values were analyzed by GraphPad Prism4 One-way ANOVA. Error bars represent standard deviations. Data shown is representative of three independent experiments with n=3 per group.

FIG. 14A-H shows Staphylococcus epidermidis modulates TRAF1 to control TLR3-dependent TNFα production. (A) S. epidermidis 10 KDa filtrate, not poly(I:C), induced negative regulator TRAF1, but neither of A20 and IRAK-M. White bar: A20; black bar: TRAF1; grey bar: IRAK-M. (b) and (c), TRAF1 was induced by S. epidermidis 10 KDa filtrate in time-dependent manner by real-time RT-PCR and western blot analyses. (d) Poly(I:C) failed to induce TRAF1 by western blot analysis. (e) Poly(I:C) recruited and activated caspase 8 to cleave TRAF1, releasing N-terminal TRAF1(N-TRAF1). Arrow: N-TRAF1. (f) Caspase 8 inhibitor prevented the cleavage of TRAF1 by poly(I:C)-activated caspase 8. Arrow: N-TRAF1. (g) Caspase 8 inhibitor completely restored the production of TLR3-dependent TNFα, which was suppressed by S. epidermidis 10 KDa filtrate. ***P<0.001. P-values were evaluated by Two-tailed t tests. n.s. no significance. (h) TRAF1 RNAi restored the production of TLR3-dependent TNFα suppressed by S. epidermidis 10 KDa filtrate. *P<0.05 and ***P<0.001. P-values were determined by using GraphPad Prism4 One-way ANOVA. Error bars represent standard deviations. Each experiment represents two independent experiments with n=3 per group.

FIG. 15A-H shows TLR2/2 ligand, Staphylococcal LTA regulates TRAF1 to suppress the production of TLR3-dependent TNFα. (a) TLR3-dependent TNFα was specifically suppressed by TLR2/2 ligand, Staphylococcus aureus LTA (LTA-SA). (b) Different LTA and factors modulate TNF production by TLRs. (c) Caspase 8 inhibitor restored the production of TLR3-dependent TNFα, which was suppressed by S. aureus LTA. n.s. no significance. (d) S. aureus LTA induced TRAF1 by western blot analysis. (e) TRAF1 RNAi partially restored LTA-inhibited TLR3-dependent TNFα by ELISA analysis. (f) and (g) show that crude LTA extracted from S. epidermidis (LYA-SE) by butonal partially suppressed TLR3-dependent TNFα expression and production. (h) S. epidermidis-derived LTA antibody prevented Staphylococcal LTA from suppressing TLR3-dependent TNFα (In progress). In b and e, **P<0.01 and ***P<0.001. P-values were evaluated by Two-tailed t tests. In a and d, *P<0.05, **P<0.01 and ***P<0.001. P-values were determined by GraphPad Prism4 One-way ANOVA. Data are the mean±s.d. of triplicate cultures and are representative of two to four independent experiments.

FIG. 16A-H shows Staphylococcus epidermidis regulates TRAF1 through TLR2 signaling to limit the production of TLR3-dependent TNFα in vivo. (a) TLR2 RNAi partially restored the production of TLR3-dependent TNFα suppressed by LTA-SA. (b) The production of TLR3-dependent TNFα suppressed by S. epidermidis 10 KDa filtrate was partially restored by TLR2 RNAi. (c) S. epidermidis 10 KDa filtrate and LTA-SA induced TRAF1 through TLR2 signaling in vivo. White bar: C57BL/6 wild-type mice; black bar: C57BL/6 TLR2-deficient mice. (d) S. epidermidis 10 KDa filtrate completely suppressed the expression of TLR3-dependent TNFα in C57BL/6 wild-type mice. (e) S. epidermidis 10 KDa filtrate totally failed to suppress the expression of TLR3-dependent TNFα in C57BL/6 TLR2^{-/-} mice. (f) LTA-SA completely suppressed the expression of TLR3-dependent TNFα in C57BL/6 wild-type mice. (g) LTA-SA partially suppressed the expression of TLR3-dependent TNFα in C57BL/6 TLR2^{-/-} mice. (h) shows animal pathophysiology images in wild-type and TLR2^{-/-} mice. (a) and (b) *P<0.05, **P<0.01 and ***P<0.001. P-values were evaluated by One-way ANOVA. c-g, *P<0.05 and **P<0.01. P-values were determined by Two-tailed t tests. n.s. no significance. Data are the mean±s.d. of four to seven stimulations and are representative of two independent experiments.

### DETAILED DESCRIPTION

As used herein and in the appended claims, the singular forms “a,” “and,” and “the” include plural referents unless the context clearly dictates otherwise. Thus, for example, reference to “a polypeptide” includes a plurality of such polypeptides and reference to “the polynucleotide” includes reference to one or more polynucleotides, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

So long as there is a need for protection against infection the evaluation of host responses that contribute to control of bacterial and viral infections is an important goal. In addition, modulating inflammation as a result of such infections as well as those resulting from autoimmune and contact with inflammatory agents is important. The disclosure provides peptides useful in treating bacterial, viral and other microbial infections. The disclosure also provides biological agents that reduce the production of pro-inflammatory mediators.

In humans, there are several classes of known antimicrobial peptides (AMPs) including α-defensins, β-defensins, and cathelicidins. There are, in addition, linear amphipathic cationic peptides from other organisms, including magainins, cecropins, dermaseptin, δ-lysin (delta haemolysin), phenol soluble modulin-delta or melittin.

Staphylococcus epidermidis and other coagulase-negative staphylococci, previously regarded as harmless contaminants, are increasingly being recognized as important pathogens and bacterial contaminants. Coagulase-negative staphylococci, the predominant species being S. epidermidis, are common pathogens in nosocomial bacteremia.

Staphylococcus epidermidis (Se) is a less common cause of opportunistic infections than S. aureus, but is still significant. S. epidermis is a major component of the skin flora and thus commonly a contaminant of cultures. S. epidermidis has been found to inhibit the growth of Staphylococcus aureus and Group A Streptococcus, the two leading causes of human skin infections and wound infections. Using biochemical and molecular biology techniques the disclosure demonstrates the biological factors produced by S. epidermidis have beneficial properties. These biological factors have effects upon microbial defense and inflammation. Accordingly, the disclosure provides methods and compositions that take advantage of the biological factors produced by S. epidermidis and other related microbes and identified herein.

The disclosure provides whole cell preparations comprising a substantially homogeneous preparation of S. epidermidis. Such a preparation can be used in the preparation of compositions for the treatment of inflammation and microbial infections. Whole cell preparation can comprise S. epidermidis or may comprise non-pathogenic (e.g., attenuated microbe) vector comprising a polypeptide as described below or an LTA agent as described below. The disclosure also provides fractions derived from such whole cells comprising a polypeptide and/or LTA agent of the disclosure. Such fractions need not comprise a purified polypeptide of the disclosure so long as it comprises a polypeptide of the disclosure and/or and LTA agent. For example, the disclosure demonstrates that a 10 kDa fraction from S. epidermidis is useful to inhibit the production of pro-inflammatory mediators.

The disclosure also provides a composition comprising a polypeptide selected from the group consisting of (i) a polypeptide comprising at least 95-99% identity to SEQ ID NO:2, 3, 4, 5, 6 or 7 and having antimicrobial activity; (ii) a polypeptide that comprises about 20-25 amino acids of SEQ ID NO: 2, 3, 4, 5, 6 or 7 and having antimicrobial activity; (iii) a polypeptide encoded by SEQ ID NO:1 or a fragment thereof; (iv) a polypeptide comprising SEQ ID NO: 2, 3, 4, 5, 6 or 7; and (v) a polypeptide consisting of SEQ ID NO: 2, 3, 4, 5, 6 or 7. Exemplary polypeptides of the disclosure include:

| | |
|---|---|
| MAADIISTIGDLVKWIIDTVNKFKK | (SEQ ID NO: 2) |
| MAQDIISTIGDLVKWIIDTVNKFKK | (SEQ ID NO: 3) |
| MAADIISTIGDLVKWIIDTVNKFTK | (SEQ ID NO: 4) |
| MAQDIISTIGDLVKWIIDTVNKFTK | (SEQ ID NO: 5) |
| MAQDIISTISDLVKWIIDTVNKFTK | (SEQ ID NO: 6) |
| MSIVSTIIEVVKTIVDIVKKFKK. | (SEQ ID NO: 7) |

In another embodiment, the disclosure provides compositions comprising one or more of the polypeptides set forth in SEQ ID NO:2-6 or 7, wherein the polypeptide comprises from about 1-10 (e.g., 2, 3, 4, 5, 6, 7, 8, or 9) conservative amino acid substitutions wherein the polypeptide has antimicrobial activity. In yet a further embodiment, the peptides can comprise non-naturally occurring amino acids. Such non-naturally occurring amino acids can be incorporated into a polypeptide of the disclosure using various solid-phase synthesis techniques. For example, solid-phase peptide synthesis using Fmoc and Boc can be used. Solid-phase peptide synthesis allows the synthesis of natural peptides which are difficult to express in bacteria, the incorporation of unnatural amino acids, peptide/protein backbone modification, and the synthesis of D-proteins, which consist of D-amino acids.

Accordingly, the polypeptides of the disclosure can comprise D-amino acids, L-amino acids, or a mixture of D- and L-amino acids. The D-form of the amino acids is particularly useful since a protein comprised of D-amino acids is expected to have a greater retention/half-life of its biological activity in vivo, because D-amino acids are not recognized by naturally occurring proteases.

In addition, alterations of the native amino acid sequence to produce variant polypeptides can be done by a variety of means known to those ordinarily skilled in the art. For instance, amino acid substitutions can be conveniently introduced into the polypeptides at the time of synthesis. Alternatively, site-specific mutations can be introduced by ligating into an expression vector a synthesized oligonucleotide comprising the modified site. Alternately, oligonucleotide-directed, site-specific mutagenesis procedures can be used and the modified polypeptide screened for activity.

It is within the skill in the art to select synthetic and naturally-occurring amino acids that effect conservative or neutral substitutions for any particular naturally-occurring amino acids. A number of factors are considered in selecting an appropriate amino acid. For example, consideration will be given to hydrophobicity or polarity of the side-chain, the general size of the side chain and the pK value of side-chains with acidic or basic character under physiological conditions. For example, lysine, arginine, and histidine are often suitably substituted for each other, and more often arginine and histidine. Similarly, glycine, alanine, valine, leucine, and isoleucine are often suitably substituted for each other. Other groups of amino acids frequently suitably substituted for each other include, but are not limited to, the group consisting of glutamic and aspartic acids; the group consisting of phenylalanine, tyrosine, and tryptophan; and the group consisting of serine, threonine, and optionally, tyrosine. Additionally, the ordinarily skilled artisan can readily group synthetic amino acids with naturally-occurring amino acids.

If desired, the polypeptides can be modified, for instance, by glycosylation, amidation, carboxylation, or phosphorylation, or by the creation of acid addition salts, amides, esters, in particular C-terminal esters, and N-acyl derivatives of the polypeptides of the disclosure. The polypeptides also can be modified to create protein derivatives by forming covalent or noncovalent complexes with other moieties in accordance with methods known in the art. Covalently-bound complexes can be prepared by linking the chemical moieties to functional groups on the side chains of amino acids comprising the polypeptides, or at the N- or C-terminus. Desirably, such modifications and conjugations do not adversely affect the activity of the polypeptides (and variants thereof). While such modifications and conjugations can have greater or lesser activity, the activity desirably is not negated and is characteristic of the unaltered polypeptide.

The polypeptides (and fragments, variants, and fusion proteins) can be prepared by any of a number of conventional techniques. The polypeptide can be isolated or purified from a naturally occurring source or from a recombinant source. For instance, in the case of recombinant proteins, a DNA fragment encoding a desired protein can be subcloned into an appropriate vector using well-known molecular genetic techniques (see, e.g., Maniatis et al., Molecular Cloning: A Laboratory Manual, 2nd ed. (Cold Spring Harbor Laboratory, 1989) and other references cited herein. The fragment can be transcribed and the protein subsequently translated in vitro. Commercially available kits also can be employed (e.g., such as manufactured by Clontech, Palo Alto, Calif.; Amersham Life Sciences, Inc., Arlington Heights, Ill.; InVitrogen, San Diego, Calif., and the like). The polymerase chain reaction optionally can be employed in the manipulation of nucleic acids.

Such polypeptides also can be synthesized using an automated peptide synthesizer in accordance with methods known in the art. Alternately, the polypeptide (and fragments, variants, and fusion proteins) can be synthesized using standard peptide synthesizing techniques well-known to those of ordinary skill in the art (e.g., as summarized in Bodanszky, Principles of Peptide Synthesis, (Springer-Verlag, Heidelberg: 1984)). In particular, the polypeptide can be synthesized using the procedure of solid-phase synthesis (see, e.g., Merrifield, J. Am. Chem. Soc., 85:2149-54 (1963); Barany et al., Int. J. Peptide Protein Res., 30:705-739 (1987); and U.S. Pat. No. 5,424,398). If desired, this can be done using an automated peptide synthesizer. Removal of the t-butyloxycarbonyl (t-BOC) or 9-fluorenylmethyloxycarbonyl (Fmoc) amino acid blocking groups and separation of the protein from the resin can be accomplished by, for example, acid treatment at reduced temperature. The polypeptide-containing mixture then can be extracted, for instance, with diethyl ether, to remove non-peptidic organic compounds, and the synthesized protein can be extracted from the resin powder (e.g., with about 25% w/v acetic acid). Following the synthesis of the polypeptide, further purification (e.g., using HPLC) optionally can be done in order to eliminate any incomplete proteins, polypeptides, peptides, or free amino acids. Amino acid and/or HPLC analysis can be performed on the synthesized polypeptide to validate its identity. For other applications according to the disclosure, it may be useful to produce the polypeptide as part of a larger fusion protein, either by chemical conjugation, or through genetic means, such as are known to those ordinarily skilled in the art. In this regard, the disclosure also provides a fusion protein comprising the isolated or purified polypeptide (or fragment thereof) or variant thereof and one or more other polypeptides/protein(s) having any desired properties or effector functions. In one aspect, the fusion polypeptide may be homodimers, trimers and the like of a polypeptide of the disclosure. In another aspect, the fusion polypeptide may be a first polypeptide of the disclosure (e.g., comprising SEQ ID NO:2) and a second polypeptide of the disclosure (e.g., comprising SEQ ID NO:7) linked to one another directly or through a linking peptide.

Accordingly, the disclosure also includes analogs, derivatives, conservative variations, and delta-haemolysin or phenol soluble modulin-delta functional fragments, provided that the analog, derivative, conservative variation, or variant has a detectable antimicrobial antibacterial and/or antiviral activity. It is not necessary that the analog, derivative, variation, or variant have activity identical to the activity of the peptide from which the analog, derivative, conservative variation, or variant is derived.

A delta haemolysin or phenol soluble modulin-delta peptide functional variant is an antimicrobial, antibacterial and/or antiviral peptide that is an altered form of a referenced delta haemolysin or phenol soluble modulin-delta, respectively. For example, the term “variant” includes a delta haemolysin or phenol soluble modulin-delta functional variant produced by the method disclosed herein in which at least one amino acid (e.g., from about 1 to 10 amino acids) of a reference peptide is substituted with another amino acid. The term “reference” peptide means any of the delta haemolysin or phenol soluble modulin-delta functional variants of the disclosure. Included within the term “derivative” is a hybrid peptide that includes at least a portion of each of two or more delta haemolysin or phenol soluble modulin-delta functional variants (e.g., 30-80% of each of two delta haemolysin or phenol soluble modulin-delta functional variants). Derivatives can be produced by adding one or a few (e.g., 1-5) amino acids to a peptide of the disclosure without completely inhibiting the activity of the peptide. In addition, C-terminal derivatives, e.g., C-terminal methyl esters, can be produced and are encompassed by the disclosure.

The disclosure also includes peptides that are conservative variations of those peptides as exemplified herein. The term “conservative variation” as used herein denotes a peptide or polypeptide in which at least one amino acid is replaced by another, biologically similar residue. Examples of conservative variations include the substitution of one hydrophobic residue, such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic for aspartic acid, or glutamine for asparagine, and the like. Neutral hydrophilic amino acids that can be substituted for one another include asparagine, glutamine, serine and threonine. The term “conservative variation” also encompasses a peptide having a substituted amino acid in place of an unsubstituted parent amino acid; typically, antibodies raised to the substituted peptide or polypeptide also specifically bind the unsubstituted peptide or polypeptide.

Delta haemolysin or phenol soluble modulin-delta functional variant variants of the disclosure can be identified by screening a large collection, or library, of random peptides or polypeptides using, for example, one of a number of animal models such as CRAMP knockout mice that display increased susceptibility to skin infections. Delta haemolysin or phenol soluble modulin-delta functional variants can be, for example, a population of peptides related in amino acid sequence to SEQ ID NO:2 or SEQ ID NO:7, respectively.

Peptide libraries include, for example, tagged chemical libraries comprising peptides and peptidomimetic molecules. Peptide libraries also comprise those generated by phage display technology. Phage display technology includes the expression of peptide molecules on the surface of phage as well as other methodologies by which a protein ligand is or can be associated with the nucleic acid which encodes it. Methods for the production of phage display libraries, including vectors and methods of diversifying the population of peptides, which are expressed, are known in the art (see, for example, Smith and Scott, Methods Enzymol. 217:228-257 (1993); Scott and Smith, Science 249:386-390 (1990); and Huse, WO 91/07141 and WO 91/07149). These or other known methods can be used to produce a phage display library, from which the displayed peptides can be cleaved and assayed for antibacterial activity. If desired, a population of peptides can be assayed for activity, and an active population can be subdivided and the assay repeated in order to isolate an active peptide from the population. Other methods for producing peptides useful in the disclosure include, for example, rational design and mutagenesis based on the amino acid sequences of a delta haemolysin or phenol soluble modulin-delta functional variant.

A delta haemolysin or phenol soluble modulin-delta functional variant can be a peptide mimetic, which is a non-amino acid chemical structure that mimics the structure of, for example, a delta haemolysin functional variant of SEQ ID NO:2 and the related peptides of SEQ ID NO:3, 4, 5, or 6 or a phenol soluble modulin-delta functional variant of SEQ ID NO:7, yet retains antimicrobial/antibacterial/antiviral activity. Such a mimetic generally is characterized as exhibiting similar physical characteristics such as size, charge or hydrophobicity in the same spatial arrangement found in the delta haemolysin or phenol soluble modulin-delta functional variant counterpart. A specific example of a peptide mimetic is a compound in which the amide bond between one or more of the amino acids is replaced, for example, by a carbon-carbon bond or other bond well known in the art (see, for example, Sawyer, Peptide Based Drug Design, ACS, Washington (1995)).

The amino acids of a delta haemolysin or phenol soluble modulin-delta, a delta haemolysin or phenol soluble modulin-delta functional variant or peptidomimetic of the disclosure are selected from the twenty naturally occurring amino acids, including, unless stated otherwise, L-amino acids and D-amino acids. The use of D-amino acids are particularly useful for increasing the life of a peptide or polypeptide. Polypeptides or peptides incorporating D-amino acids are resistant to proteolytic digestion. The term amino acid also refers to compounds such as chemically modified amino acids including amino acid analogs, naturally occurring amino acids that are not usually incorporated into proteins such as norleucine, and chemically synthesized compounds having properties known in the art to be characteristic of an amino acid, provided that the compound can be substituted within a peptide such that it retains its biological activity. For example, glutamine can be an amino acid analog of asparagine, provided that it can be substituted within an active fragment of a delta haemolysin or phenol soluble modulin-delta functional variant and the like such that it retains its antimicrobial/antibacterial/antiviral activity. Other examples of amino acids and amino acids analogs are listed in Gross and Meienhofer, The Peptides: Analysis, Synthesis, Biology, Academic Press, Inc., New York (1983). An amino acid also can be an amino acid mimetic, which is a structure that exhibits substantially the same spatial arrangement of functional groups as an amino acid but does not necessarily have both the “-amino” and “-carboxyl” groups characteristic of an amino acid.

The activity of the peptides of the disclosure can be determined using conventional methods known to those of skill in the art, such as in a “minimal inhibitory concentration (MIC)”, whereby the lowest concentration at which no change in OD is observed for a given period of time is recorded as the MIC. Alternatively, a “fractional inhibitory concentration (FIC)” assay can be used to measure synergy between the peptides of the disclosure, or the peptides in combination with known antibiotics. FICs can be performed by checkerboard titrations of peptides in one dimension of a microtiter plate, and of antibiotics in the other dimension, for example. The FIC is a function of the impact of one antibiotic on the MIC of the other and vice versa. A FIC of 1 indicates that the influence of the compounds is additive and a FIC of less than 1 indicates that the compounds act synergistically.

The disclosure also includes isolated polynucleotides (e.g., DNA, cDNA, or RNA) encoding the peptides of the disclosure. Included are polynucleotides that encode analogs, mutants, conservative variations, and variants of the peptides described herein. The term “isolated” as used herein refers to a polynucleotide that is substantially free of proteins, lipids, and other polynucleotides with which an in vivo-produced polynucleotide naturally associates. Typically, the polynucleotide is at least 70%, 80%, or 90% isolated from other matter, and conventional methods for synthesizing polynucleotides in vitro can be used in lieu of in vivo methods. As used herein, “polynucleotide” refers to a polymer of deoxyribonucleotides or ribonucleotides, in the form of a separate fragment or as a component of a larger genetic construct (e.g., by operably linking a promoter to a polynucleotide encoding a peptide of the disclosure). Numerous genetic constructs (e.g., plasmids and other expression vectors) are known in the art and can be used to produce the peptides of the disclosure in cell-free systems or prokaryotic or eukaryotic (e.g., yeast, insect, or mammalian) cells. By taking into account the degeneracy of the genetic code, one of ordinary skill in the art can readily synthesize polynucleotides encoding the peptides of the disclosure. The polynucleotides of the disclosure can readily be used in conventional molecular biology methods to produce the peptides of the disclosure.

In one embodiment, a delta haemolysin or phenol soluble modulin-delta functional variant polynucleotide/nucleic acid of the disclosure comprises a sequence that encodes SEQ ID NO:2, 3, 4, 5, 6 or 7. In another aspect, the delta haemolysin polynucleotide comprises SEQ ID NO:1.

The disclosure also includes polynucleotides useful for generating a polypeptide of the disclosure. In addition, such polynucleotides can be used for in vivo production of a polypeptide of the disclosure (e.g., by gene delivery techniques). Polynucleotides encoding such polypeptides can be generated using codons corresponding to each amino acid in the polypeptide chain. As one of skill in the art will recognized the degeneracy of the genetic code can result in different codons providing for the same amino acid upon translation. A polynucleotide encoding SEQ ID NO:2 can comprise, for example, 5′-ttattttttg aatttattaa ctgtatcgat aatccatttt actaaatcac cgattgtaga aatgatatct gctgccat-3′ (SEQ ID NO:1).

As used herein, “nucleic acid” or “polynucleotide” refers to a polymer of deoxyribonucleotides or ribonucleotides, in the form of a separate fragment or as a component of a larger genetic construct (e.g., by operably linking a promoter to a nucleic acid encoding a peptide of the disclosure). Numerous genetic constructs (e.g., plasmids and other expression vectors) are known in the art and can be used to produce the peptides of the disclosure in cell-free systems or prokaryotic or eukaryotic (e.g., yeast, insect, or mammalian) cells. By taking into account the degeneracy of the genetic code, one of ordinary skill in the art can readily synthesize nucleic acids encoding the polypeptides of the disclosure. The nucleic acids of the disclosure can readily be used in conventional molecular biology methods to produce the peptides of the disclosure.

Polynucleotides encoding the delta haemolysin or phenol soluble modulin-delta or a functional variant of the disclosure can be inserted into an “expression vector.” The term “expression vector” refers to a genetic construct such as a plasmid, virus or other vehicle known in the art that can be engineered to contain a polynucleotide encoding a peptide or polypeptide of the disclosure. Such expression vectors are typically plasmids that contain a promoter sequence that facilitates transcription of the inserted genetic sequence in a host cell. The expression vector typically contains an origin of replication, and a promoter, as well as genes that allow phenotypic selection of the transformed cells (e.g., an antibiotic resistance gene). Various promoters, including inducible and constitutive promoters, can be utilized in the disclosure. Typically, the expression vector contains a replicon site and control sequences that are derived from a species compatible with the host cell.

Transformation or transfection of a host cell with a polynucleotide of the disclosure can be carried out using conventional techniques well known to those skilled in the art. For example, where the host cell is E. coli, competent cells that are capable of DNA uptake can be prepared using the CaCl₂, MgCl₂ or RbCl methods known in the art. Alternatively, physical means, such as electroporation or microinjection can be used. Electroporation allows transfer of a polynucleotide into a cell by high voltage electric impulse. Additionally, polynucleotides can be introduced into host cells by protoplast fusion, using methods well known in the art. Suitable methods for transforming eukaryotic cells, such as electroporation and lipofection, also are known.

“Host cells” encompassed by of the disclosure are any cells in which the polynucleotides of the disclosure can be used to express the delta haemolysin or phenol soluble modulin-delta or a functional variant thereof of the disclosure. The term also includes any progeny of a host cell. Host cells, which are useful, include bacterial cells (e.g., attenuated bacterial vectors), fungal cells (e.g., yeast cells), plant cells and animal cells. For example, host cells can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., Battey, I., Basic Methods in Molecular Biology (1986)). As representative examples of appropriate hosts, there may be mentioned: fungal cells, such as yeast; insect cells such as Drosophila S2 and Spodoptera Sf9; animal cells such as CHO, COS or Bowes melanoma; plant cells, and the like. The selection of an appropriate host is deemed to be within the scope of those skilled in the art from the teachings herein.

Host cells can be eukaryotic host cells (e.g., mammalian cells). In one aspect, the host cells are mammalian production cells adapted to grow in cell culture. Examples of such cells commonly used in the industry are CHO, VERO, BHK, HeLa, CV1 (including Cos; Cos-7), MDCK, 293, 3T3, C127, myeloma cell lines (especially murine), PC12 and W138 cells. Chinese hamster ovary (CHO) cells are widely used for the production of several complex recombinant proteins, e.g. cytokines, clotting factors, and antibodies (Brasel et al., Blood 88:2004-2012, 1996; Kaufman et al., J. Biol Chem 263: 6352-6362, 1988; McKinnon et al., J Mol Endocrinol 6:231-239, 1991; Wood et al., J. Immunol. 145:3011-3016, 1990). The dihydrofolate reductase (DHFR)-deficient mutant cell lines (Urlaub et al., Proc Natl Acad Sci USA 77:4216-4220, 1980) are the CHO host cell lines commonly used because the efficient DHFR selectable and amplifiable gene expression system allows high level recombinant protein expression in these cells (Kaufman, Meth Enzymol 185:527-566, 1990). In addition, these cells are easy to manipulate as adherent or suspension cultures and exhibit relatively good genetic stability. CHO cells and recombinant proteins expressed in them have been extensively characterized and have been approved for use in clinical manufacturing by regulatory agencies.

Polynucleotides encoding the peptides of the disclosure can be isolated from a cell (e.g., a cultured cell), or they can be produced in vitro. A DNA sequence encoding a delta haemolysin or phenol soluble modulin-delta or a functional variant thereof of interest can be obtained by: 1) isolation of a double-stranded DNA sequence from genomic DNA; 2) chemical manufacture of a polynucleotide such that it encodes the delta haemolysin or phenol soluble modulin-delta or functional variant of interest; or 3) in vitro synthesis of a double-stranded DNA sequence by reverse transcription of mRNA isolated from a donor cell (i.e., to produce cDNA).

Included in the disclosure are polynucleotides that encode analogs, mutants, conservative variations, and variants of the peptides described herein. For example, an isolated polynucleotide encoding a delta haemolysin functional variant of the disclosure can comprise the sequence of SEQ ID NO:1 comprising at least one (typically 2, 3, 4, 5, 6, 7, 8, 9, or 10) mutations that can result in a peptide with increased stability, activity or that results in a silent mutation.

Such polynucleotides include naturally occurring, synthetic, and intentionally manipulated polynucleotides. For example, a delta haemolysin or phenol soluble modulin-delta or variant polynucleotide may be subjected to site-directed mutagenesis. A delta haemolysin or phenol soluble modulin-delta or variant polynucleotide includes sequences that are degenerate as a result of the genetic code. There are 20 natural amino acids, most of which are specified by more than one codon. Therefore, all degenerate nucleotide sequences are included so long as the amino acid sequence of a delta haemolysin or phenol soluble modulin-delta or variant encoded by the nucleotide sequence is functionally unchanged. Accordingly, a polynucleotide of the disclosure includes (i) a polynucleotide encoding a delta haemolysin or phenol soluble modulin-delta or functional variant (e.g., SEQ ID NO:2, 3, 4, 5, 6 or 7); (ii) a polynucleotide encoding SEQ ID NO:1 or a variant thereof; (iii) a polynucleotide of (ii), wherein T is U; and (iv) a polynucleotide comprising a sequence that is complementary to (ii) and (iv) above. A “polynucleotide” of the disclosure also includes those polynucleotides capable of hybridizing, under stringent hybridization conditions, to sequences of (i)-(iv), above. “Stringent hybridization conditions” refers to an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. It will be recognized that a polynucleotide of the disclosure, may be operably linked to a second heterologous polynucleotide such as a promoter or a heterologous sequence encoding a desired peptide or polypeptide sequence.

Any of various art-known methods for protein purification can be used to isolate the peptides of the disclosure. For example, preparative chromatographic separations and immunological separations (such as those employing monoclonal or polyclonal antibodies) can be used. Carrier peptides can facilitate isolation of fusion proteins that include the peptides of the disclosure. Purification tags can be operably linked to a delta haemolysin or phenol soluble modulin-delta or functional variant of the disclosure. For example, glutathione-S-transferase (GST) allows purification with a glutathione agarose affinity column. When either Protein A or the ZZ domain from Staphylococcus aureus is used as the tag, purification can be accomplished in a single step using an IgG-sepharose affinity column. The pOprF-peptide, which is the N-terminal half of the P. aeruginosa outer membrane protein F, can readily be purified because it is the prominent protein species in outer membrane preparations. If desired, the fusion peptides can be purified using reagents that are specifically reactive with (e.g., specifically bind) the delta haemolysin or phenol soluble modulin-delta or functional variant of the fusion peptide. For example, monoclonal or polyclonal antibodies that specifically bind the delta haemolysin or phenol soluble modulin-delta or functional variant can be used in conventional purification methods. Techniques for producing such antibodies are well known in the art.

A fusion construct comprising a peptide or polypeptide linked to a delta haemolysin or phenol soluble modulin-delta or functional variant of the disclosure can be linked at either the amino or carboxy terminus of the peptide. Typically, the polypeptide that is linked to the delta haemolysin or phenol soluble modulin-delta or functional variant is sufficiently anionic or cationic such that the charge associated with the delta haemolysin or phenol soluble modulin-delta or functional variant is overcome and the resulting fusion peptide has a net charge that is neutral or negative. The peptide or polypeptide linked to a peptide of the disclosure can correspond in sequence to a naturally-occurring protein or can be entirely artificial in design. Functionally, the polypeptide linked to a delta haemolysin or phenol soluble modulin-delta or functional variant (the “carrier polypeptide”) may help stabilize the delta haemolysin or phenol soluble modulin-delta or functional variant and protect it from proteases, although the carrier polypeptide need not be shown to serve such a purpose. Similarly, the carrier polypeptide may facilitate transport of the fusion peptide. Examples of carrier polypeptides that can be utilized include anionic pre-pro peptides and anionic outer membrane peptides. The disclosure is not limited to the use of these carrier polypeptides; others suitable carrier polypeptides are known to those skilled in the art. In another aspect, a linker moiety comprising a protease cleavage site may be operably linked to a delta haemolysin or phenol soluble modulin-delta or functional variant of the disclosure. For example, the linker may be operable between the domains of a fusion protein (e.g., a fusion protein comprising a delta haemolysin or phenol soluble modulin-delta or functional variant and a carrier polypeptide). Because protease cleavage recognition sequences generally are only a few amino acids in length, the linker moiety can include the recognition sequence within flexible spacer amino acid sequences, such as GGGGS (SEQ ID NO:8). For example, a linker moiety including a cleavage recognition sequence for Adenovirus endopeptidase could have the sequence GGGGGGSMFG GAKKRSGGGG GG (SEQ ID NO:9). If desired, the spacer DNA sequence can encode a protein recognition site for cleavage of the carrier polypeptide from the delta haemolysin or phenol soluble modulin-delta or functional variant. Examples of such spacer DNA sequences include, but are not limited to, protease cleavage sequences, such as that for Factor Xa protease, the methionine, tryptophan and glutamic acid codon sequences, and the pre-pro defensin sequence. Factor Xa is used for proteolytic cleavage at the Factor Xa protease cleavage sequence, while chemical cleavage by cyanogen bromide treatment releases the peptide at the methionine or related codons. In addition, the fused product can be cleaved by insertion of a codon for tryptophan (cleavable by o-iodosobenzoic acid) or glutamic acid (cleavable by Staphylococcus protease). While insertion of such spacer oligonucleotides is not a requirement for the production of delta haemolysin or phenol soluble modulin-delta or functional variant, such oligonucleotides can enhance the stability of the fusion polypeptide.

The term “antimicrobial” as used herein means that the peptide destroys, or inhibits or prevents the growth or proliferation of, a microbe (e.g., a bacterium, fungus, and/or virus). Likewise, the term “antiviral” as used herein means that a peptide destroys, or inhibits or prevents the growth or proliferation of a virus or a virus-infected cell. The term “anti-tumor” as used herein means that a peptide prevents, inhibits the growth of, or destroys, a tumor cell(s). Similarly, the term “antifungal” means that a peptide prevents, destroys, or inhibits the growth of a fungus. The term “anti-inflammatory” means that the biological agent reduces signs, symptoms of production of biological proinflammatory mediators. For example, an LTA biological agent reduces the biological effects or production of the proinflammatory mediator TNFα.

The term “purified” and “substantially purified” as used herein refers to a polypeptide, peptide or biological agent (e.g., LTA) that is substantially free of other proteins, lipids, and polynucleotides (e.g., cellular components with which an in vivo-produced polypeptide or peptide would naturally be associated). Typically, the peptide is at least 70%, 80%, or most commonly 90% pure by weight.

The disclosure demonstrates that delta-haemolysin and/or phenol soluble modulin-delta and functional fragments thereof, have antimicrobial and antiviral activity in vitro and in vivo. The mechanisms by which a delta-haemolysin or phenol soluble modulin-delta of the disclosure kills bacteria and fungi can be through binding of the peptide to the microbial cell membrane, after which the membrane's proton gradient and integrity are lost.

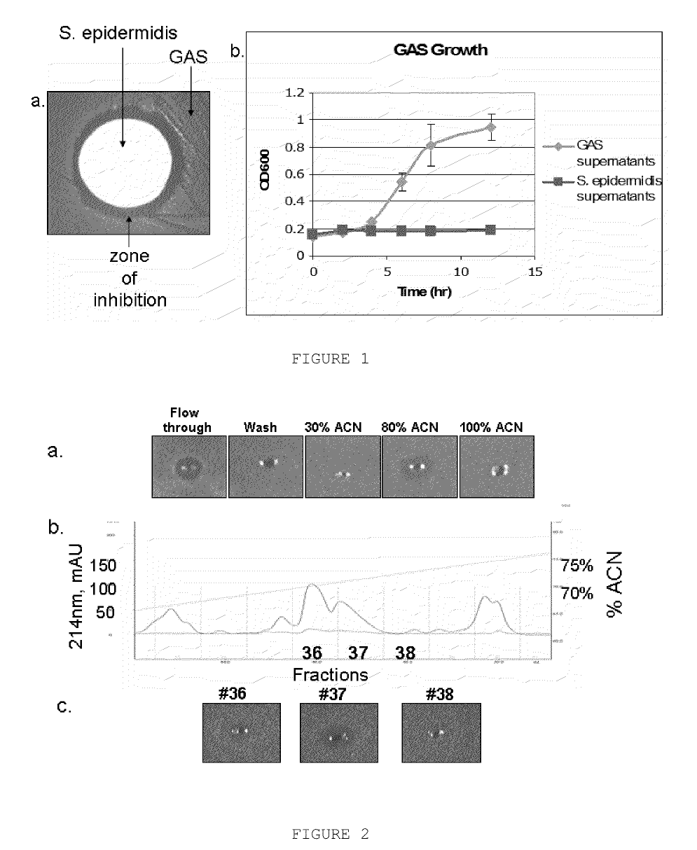

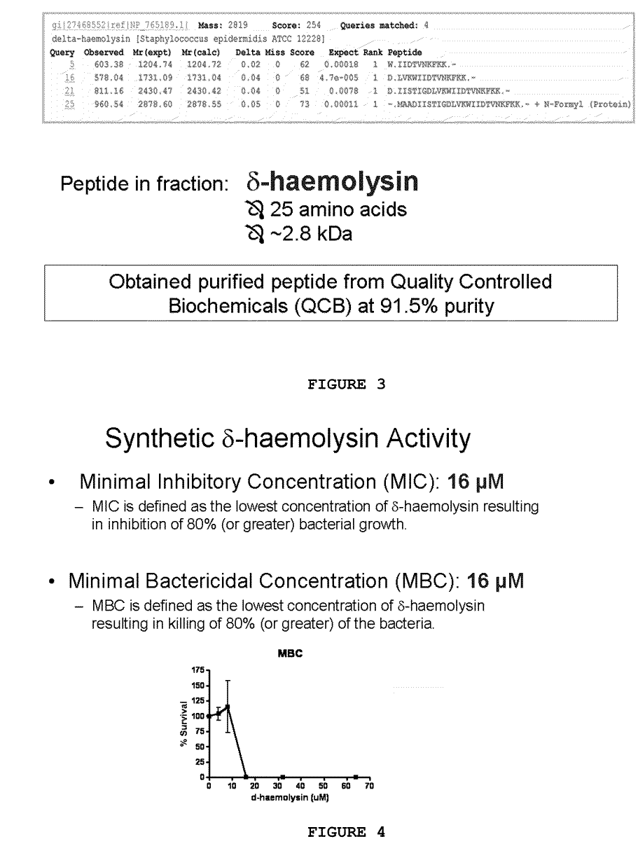

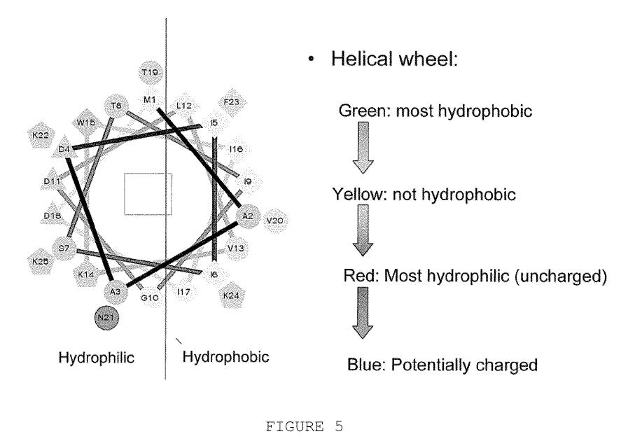

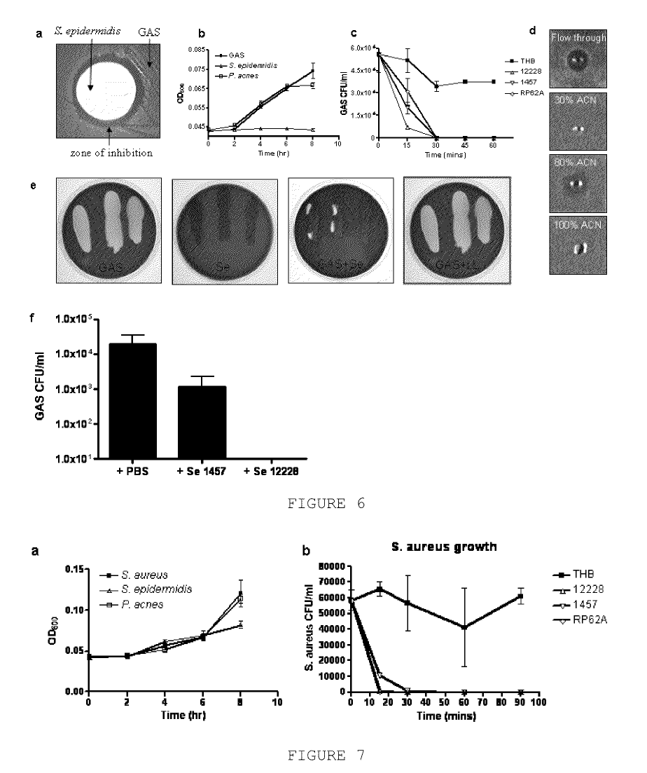

The disclosure demonstrates that the presence of Se on normal skin inhibits Group A Streptococcus (GAS) survival when compared to skin that had been previously sanitized with alcohol. Growth of GAS on agar media was also inhibited upon co-culture with Se (FIG. 1). GAS growth in THB media as measured by OD₆₀₀ and on agar as measured by radial diffusion (clear zone of 28.26 mm²), was inhibited by the addition of cell-free culture supernatants prepared from Se. Inhibition of GAS due to Se conditioned supernatants was statistically significant at 6, 8, and 12 hours (p<0.001) compared to growth in the absence of Se or conditioned supernatants prepared from other bacteria such as Staphylococcus aureus. A factor present in Se was hypothesized to be the inhibitor. In initial studies, supernatant from conditioned Se was purified by HPLC and antimicrobial activity identified by radial diffusion assay (FIG. 2). Maximal activity eluted at 72% acetonitrile/0.1% trifluoroacetic acid (clear zone of 17.86 mm²). MALDI TOF-TOF identified a peptide in this fraction known as delta-haemolysin, a membrane active peptide of unknown function in Se (FIG. 3). Synthetic delta-haemolysin had an MIC and MBC of 16 μM when tested with GAS (FIG. 4). Subsequent studies, described in further detail below, showed that there was an initial additional inhibitory factor present in Se (e.g., phenol soluble modulin-delta). Overall, these studies show a role of Se in cutaneous protection against infection, a first line of defense of the skin is the resident microflora itself.

This disclosure makes use of the normal bacteria that live on the skin to protect against disease causing bacteria. Because of its natural abundance it is predicted to be safe, effective, and inexpensive to make. Although S. epidermidis produces antimicrobial peptides, delta-haemolysin and phenol soluble modulin-delta are not implicated and the application of these factors to protect against human infections is useful.

Accordingly, delta-haemolysin and/or phenol soluble modulin-delta alone or an extract of S. epidermidis comprising delta-haemolysin and/or phenol soluble modulin-delta, can be applied topically or administered systemically to reduce the severity of infection caused by microbes such as Staphylococcus and Group A Streptococcus.

Treatment of infectious skin disorders with delta-haemolysin and/or phenol soluble modulin-delta and S. epidermidis result in clearance of infection caused by Group A Streptococcus or Staphylcoccus aureus. Topical application would decrease recovery time from bacterial skin infections in wounds, diabetic ulcers, acne, rosacea, atopic dermatitis, pyodermas, burn wounds, catheter infections, and other dermatological diseases. In addition, it could be used systemically for treatment of Group A Streptococcus infections in normal and immunocompromised patients.

The disclosure also provides a method for inhibiting the growth of a bacterium by contacting the bacterium with an inhibiting effective amount of a delta haemolysin or phenol soluble modulin-delta or a functional variant of the disclosure. The term “contacting” refers to exposing the bacterium to a delta haemolysin or phenol soluble modulin-delta or a functional variant peptide so that the peptide can inhibit, kill, or lyse bacteria. Contacting of an organism with a delta haemolysin or phenol soluble modulin-delta or functional variant of the disclosure can occur in vitro, for example, by adding the peptide to a bacterial culture to test for susceptibility of the bacteria to the peptide, or contacting a bacterially contaminated surface with the peptide. Alternatively, contacting can occur in vivo, for example by administering the peptide to a subject afflicted with a bacterial infection or susceptible to infection. In vivo contacting includes both parenteral as well as topical. “Inhibiting” or “inhibiting effective amount” refers to the amount of peptide that is sufficient to cause, for example, a bacteriostatic or bactericidal effect. Bacteria that can be affected by the peptides of the disclosure include both gram-negative and gram-positive bacteria. For example, bacteria that can be affected include Staphylococcus aureus, Streptococcus pyogenes (group A), Streptococcus sp. (viridans group), Streptococcus agalactiae (group B), S. bovis, Streptococcus (anaerobic species), Streptococcus pneumoniae, and Enterococcus sp.; Gram-negative cocci such as, for example, Neisseria gonorrhoeae, Neisseria meningitidis, and Branhamella catarrhalis; Gram-positive bacilli such as Bacillus anthracis, Bacillus subtilis, P. acne Corynebacterium diphtheriae and Corynebacterium species which are diptheroids (aerobic and anerobic), Listeria monocytogenes, Clostridium tetani, Clostridium difficile, Escherichia coli, Enterobacter species, Proteus mirablis and other sp., Pseudomonas aeruginosa, Klebsiella pneumoniae, Salmonella, Shigella, Serratia, and Campylobacter jejuni. Infection with one or more of these bacteria can result in diseases such as bacteremia, pneumonia, meningitis, osteomyelitis, endocarditis, sinusitis, arthritis, urinary tract infections, tetanus, gangrene, colitis, acute gastroenteritis, impetigo, acne, acne posacue, wound infections, born infections, fascitis, bronchitis, and a variety of abscesses, nosocomial infections, and opportunistic infections. The method for inhibiting the growth of bacteria can also include contacting the bacterium with the peptide in combination with one or more antibiotics or other bacteriostatics (e.g., cathelicidins or amphipathic cationic peptides).

Fungal organisms may also be affected by the delta haemolysin or phenol soluble modulin-delta or functional variants of the disclosure and include dermatophytes (e.g., Microsporum canis and other Microsporum sp.; and Trichophyton sp. such as T. rubrum, and T. mentagrophytes), yeasts (e.g., Candida albicans, C. Tropicalis, or other Candida species), Saccharomyces cerevisiae, Torulopsis glabrata, Epidermophyton floccosum, Malassezia furfur (Pityropsporon orbiculare, or P. ovale), Cryptococcus neoformans, Aspergillus fumigatus, Aspergillus nidulans, and other Aspergillus sp., Zygomycetes (e.g., Rhizopus, Mucor), Paracoccidioides brasiliensis, Blastomyces dermatitides, Histoplasma capsulatum, Coccidioides immitis, and Sporothrix schenckii.

Commensal bacteria can modulate epithelial proinflammatory responses by releasing proteinases to cleave and inactivate cytokines in the guts. Using the methods described below, agents useful for modifying proinflammatory responses were identified. The disclosure identifies a pathway that prevents the overproduction of Toll-like receptor (TLR) 3-dependent tumor necrosis factor-alpha (TNFα) by skin inhabitant Staphylococcus epidermidis and provides a Staphylococcal LTA and compositions thereof as TNFα inhibitor. Through TLR2 signaling pathway Staphylococcus epidermidis induced a negative regulator of TLR3, tumor necrosis factor receptor (TNFR)-associated factor 1 (TRAF1), whereas poly(I:C) triggered TLR3 to overexpress TIR domain-containing adapter inducing IFN-beta (TRIF) to recruit and activate caspase 8, resulting in the cleavage of TRAF1. The released N-terminal TRAF1 was required for turning off TLR3 signaling to limit the production of the proinflammatory cytokine TNFα. These disclosure demonstrates a critical pathway in skin that TLR2-TLR3 cross-talk controls TRAF1 against inflammations caused by viruses and highlight the therapeutic potential of partially antagonizing the TLR3 pathway by Staphylococcus epidermidis.

Inflammation is a key element of the innate immune system in the response to a variety of challenges, including those caused by bacterial and viral infections as well as by damaged or dying host cells. Excessive uncontrolled inflammation results in a variety of pathological conditions and evolution of the inflammatory responses is thus a result of a trade-off between its beneficial and detrimental effects. To limit inflammation several negative regulators of TLR signaling are involved via sequestration of signaling molecules, blockade of their recruitment, degradation of target proteins or inhibition of transcription. In vitro, macrophages from A20-deficient mice have increased production of proinflammatory cytokines after stimulation with the TLR2, TLR3 and TLR9 ligands. In vivo, IRAKM-deficient mice have greater inflammatory responses to bacterial infection than do wild-type mice. Furthermore, to control inflammation commensal bacteria modulated epithelial proinflammatory responses by releasing proteinases to cleave and inactivate cytokines IL-1 and IL-6 or interference with signaling by inhibition of IκB ubiquitination in gut. The disclosure demonstrates that S. epidermidis controls TRAF1 to suppress the excessive production of TNFα caused by TLR3 ligand through TLR2-TLR3 cross-talk in the skin.

The disclosure thus provides an LTA agent useful for the treatment of inflammatory disease and disorders. An LTA agent refers to a lipoteichoic acid containing composition or fraction (e.g., a 10 kDa fraction from S. epidermidis) obtained from a microbial organism.

LTA comprises a polymer chain of polyglycerophosphate as a backbone structure and glycolipids of cytoplasmic membrane origin. See Wicken et al. (1977) Biological Properties of Lipoteichoic Acids, Microbiology, pp. 360-365. Electronmicroscopy reveals that one end of LTA is linked to cytoplasmic membrane glycolipid while the other end extends to the cell outer surface of the bacteria through the cell wall peptidoglycan layer.

The lipoteichoic acid or a fraction comprising a LTA is from at least one gram-positive organism which may belong to Streptococcus, Micrococcus, Lactobacillus, Staphylococcus, Bacillus, or Listeria. Typically, the gram-positive organism is S. aureus, S. epidermidis, S. pyogenes, N. cereus, L. monocytogenes, or belongs to groups A, B, C, or G of Streptococcus. In some embodiments, the gram-positive organism belongs to group A Streptococcus.

Examples of gram-positive bacteria having LTA include those belonging to the genera such as Streptococcus, Micrococcus, Lactobacillus, Staphylococcus, Bacillus, and Listeria. The preparation of LTA from whole cells, or a cell envelope fraction of these bacteria may be obtained, for example, according to the method described by Beachey et al. (1979) Infect. Immun. 23:618-625, which is herein incorporated by reference.

The highly conserved nature of LTA obviates the need to use strain specific LTA. LTA may be used as a an anti-inflammatory agent alone or in combination with one or more therapeutic agents (e.g., a TNFR fragment or receptor) or LTA may be conjugated to a carrier protein or incorporated in liposomes by standard techniques known in the art.

Because LTA is amphipathic, it is soluble in both water and lipophilic medium and may be formulated by a conventional formulation process into any desired form.

The disclosure demonstrates that an LTA agent is useful in reducing the production of proinflammatory mediators such as Tumor Necrosis Factor. Tumor Necrosis Factor (TNF) and interleukin-1 (IL-1) are cytokines that have been implicated in a wide range of biological processes, including inflammation. The recruitment of immune cells to sites of injury involves the concerted interactions of a large number of soluble mediators. Such cytokines are derived from mononuclear cells and macrophages, along with other cell types. Physiologically, they produce many of the same proinflammatory responses, including fever, sleep and anorexia, mobilization and activation of polymorphonuclear leukocytes, induction of cyclooxygenase and lipoxygenase enzymes, increase in adhesion molecule expression, activation of B-cells, T-cells and natural killer cells, and stimulation of production of other cytokines. Such cytokines play key roles in a large number of pathological conditions, including rheumatoid arthritis, septic shock, inflammatory bowel disease, bone mass loss, cancer, dermal sensitization disorders, diabetes, obesity and neurological conditions such as ischemic stroke, closed-head injuries, psoriasis and the like. Various factors are modulated upon cytokine production including members of the NF-κB family of transcription factors (see, e.g., FIG. 17).

The best studied member of this family of transcription factors is NF-κB, which generally exists in cells as a heterodimer of two proteins: p50 (NF-κB1) and p65 (Re1A). NF-κB, in its inactive form, resides in the cytoplasm of cells. In response to various types of stimuli, such as proinflammatory cytokines (e.g., TNF and IL-1), ultraviolet irradiation and viral infection NF-κB migrates to the nucleus.

Referring to FIG. 17, the disclosure demonstrates that LTA agents (e.g., LTA containing fractions or LTA) result in inhibition of the cascade leading to NF-κB activation and thereby production of pro-inflammatory mediators.

In one aspect, Staphylococcus epidermidis and a S. epidermidis 10 ka filtrate act as a TNF-alpha inhibitor to prevent, ameliorate and treat diseases caused by abnormal production of TNF-alpha without decreasing innate immunity. Staphylococcus epidermidis and S. epidermidis 10 ka filtrate modulate tumor necrosis factor receptor associated factor 1 (TRAF1) to bind to TIR domain-containing adapter inducing IFN-beta (TRIF), and then shutting off toll-like receptor 3 (TLR3) signaling, resulting in the inhibition of tumor necrosis factor-alpha (TNF-alpha) production in epithelial cells. Once these cells are treated they have increased expression of antimicrobial peptides than before treatment. Consequently, treatment of these cells leads to the reduction of inflammation without decreasing innate immune responses.

Hence, Staphylococcus epidermidis and S. epidermidis 10 ka filtrate can be used to treat or prevent any inflammatory disease or autoimmune disorder caused by excessive production of TNF-alpha. Exemplary diseases which can be treated by Staphylococcus epidermidis and S. epidermidis 10 ka filtrate include psoriasis, rheumatoid arthritis, septic shock, and Crohn's disease. In addition, treatment of skin infections caused by bacteria, viruses, and the like, will benefit from enhanced innate immune responses due to treatment with Staphylococcus epidermidis and S. epidermidis 10 ka filtrate.

Accordingly, the disclosure provide methods and compositions useful for modulating inflammation in a subject by contacting the subject with an effective amount of an LTA-agent of the disclosure.

A peptide(s) of the disclosure can be administered to any host, including a human or non-human animal, in an amount effective to inhibit growth of a bacterium, virus, and/or fungus. Thus, the peptides are useful as antimicrobial agents, antiviral agents, and/or antifungal agents. An LTA agent of the disclosure can be administered to any host, including a human or non-human animal, in an amount effective to inhibit inflammation of the activity or production of a pro-inflammatory mediator.

Any of a variety of art-known methods can be used to administer a polypeptide, peptide or LTA agent to a subject. For example, the polypeptide, peptide or LTA agent of the disclosure can be administered parenterally by injection or by gradual infusion over time. The polypeptide, peptide or LTA agent can be administered intravenously, intraperitoneally, intramuscularly, subcutaneously, intracavity, or transdermally.

In another aspect, a delta haemolysin or phenol soluble modulin-delta or functional variant or an LTA agent of the disclosure may be formulated for topical administration (e.g., as a lotion, cream, spray, gel, or ointment). Such topical formulations are useful in treating or inhibiting microbial, fungal, viral presence or infections or inflammation on the eye, skin, and mucous membranes such as mouth, nasal-pharynx, intestine/rectum, vagina and the like. Examples of formulations in the market place include topical lotions, creams, soaps, wipes, and the like. It may be formulated into liposomes to reduce toxicity or increase bioavailability. Other methods for delivery of the polypeptide, peptide or LTA agent include oral methods that entail encapsulation of the polypeptide, peptide or LTA agent in microspheres or proteinoids, aerosol delivery (e.g., to the lungs), or transdermal delivery (e.g., by iontophoresis or transdermal electroporation). Other methods of administration will be known to those skilled in the art.

Preparations for parenteral administration of a polypeptide, peptide or LTA agent of the disclosure include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, anti-oxidants, cheating agents, inert gases and the like also can be included.

The disclosure provides a method for inhibiting a topical bacterial, viral and/or fungal-associated disorder by contacting or administering a therapeutically effective amount of a polypeptide or peptide of the disclosure to a subject who has, or is at risk of having, such a disorder. In another embodiment, the disclosure provides a method for inhibiting inflammation or an inflammatory disorder of the epidermis comprising contacting or administering a therapeutically effective amount of an LTA agent of the disclosure to a subject who has, or is at risk of having such a disorder. The term “inhibiting” means preventing or ameliorating a sign or symptoms of a disorder (e.g., a rash, sore, and the like). Examples of disease signs that can be ameliorated include an increase in a subject's blood level of TNF, fever, hypotension, neutropenia, leukopenia, thrombocytopenia, disseminated intravascular coagulation, adult respiratory distress syndrome, shock, and organ failure. Examples of subjects who can be treated in the disclosure include those at risk for, or those suffering from, a toxemia, such as endotoxemia resulting from a gram-negative bacterial infection, venom poisoning, or hepatic failure. Other examples include subjects having a dermatitis, a psoriasis as well as those having skin infections or injuries subject to infection with gram-positive or gram-negative bacteria, a virus, or a fungus. Examples of candidate subjects include those suffering from infection by E. coli, Hemophilus influenza B, Neisseria meningitides, staphylococci, or pneumococci. Other patients include those suffering from gunshot wounds, renal or hepatic failure, trauma, burns, immunocompromising infections (e.g., HIV infections), hematopoietic neoplasias, multiple myeloma, Castleman's disease or cardiac myxoma. Those skilled in the art of medicine can readily employ conventional criteria to identify appropriate subjects for treatment in accordance with the disclosure.

The term “therapeutically effective amount” as used herein for treatment of a subject afflicted with a disease or disorder means an amount of delta haemolysin, phenol soluble modulin-delta or a functional variants, or an LTA agent sufficient to ameliorate a sign or symptom of the disease or disorder. For example, a therapeutically effective amount can be measured as the amount sufficient to decrease a subject's symptoms of dermatitis or rash by measuring the frequency of severity of skin sores. Typically, the subject is treated with an amount to reduce a symptom of a disease or disorder by at least 50%, 90% or 100%. Generally, the optimal dosage of the polypeptide or peptide will depend upon the disorder and factors such as the weight of the subject, the type of bacteria, virus or fungal infection, the sex of the subject, and degree of symptoms. Nonetheless, suitable dosages can readily be determined by one skilled in the art. Typically, a suitable dosage is 0.5 to 40 mg peptide/kg body weight, e.g., 1 to 8 mg peptide/kg body weight.

A “viral killing amount” is an amount sufficient to achieve a virus-killing blood concentration or a viral-killing surface concentration in or on the patient or subject receiving the treatment. In accordance with its conventional definition, an “antiviral agent,” as used herein, is a chemical or biologic substance that inhibits the growth of, spread of, or kills viral particles.

If desired, a suitable therapy regime can combine administration of a polypeptide, peptide or LTA agent of the disclosure with one or more additional therapeutic agents (e.g., an antibacterial peptide such as a cathelicidin polypeptide, an inhibitor of TNF, an antibiotic, and the like). The peptide(s), other therapeutic agents, and/or antibiotic(s) can be administered, simultaneously, but may also be administered sequentially. Suitable antibiotics include aminoglycosides (e.g., gentamicin), beta-lactams (e.g., penicillins and cephalosporins), quinolones (e.g., ciprofloxacin), and novobiocin. A combination therapy can comprise a delta haemolysin or phenol soluble modulin-delta or functional variant and a cathelicidin polypeptide. Such a cathelicidin polypeptide can comprise the N-terminal cathelin-like fragment, or the C-terminal domain of cathelicidin can be co-administered or administered sequentially (see, e.g., U.S. Pat. No. 7,173,007, which is incorporated herein by reference in its entirety).

Cathelicidin proteins are composed of two distinct domains: an N-terminal “cathelin-like” or “prosequence” domain and the C-terminal domain of the mature AMP. The C-terminal domains of cathelicidins were among the earliest mammalian AMPs to show potent, rapid, and broad-spectrum killing activity. The term “cathelin-like” derives from the similarity of the N-terminal sequence with that of cathelin, a 12 kDa protein isolated from porcine neutrophils that shares similarity with the cystatin superfamily of cysteine protease inhibitors.

The structure of the N-terminal 96-104 residue protein domain (the N-terminal cathelin-like domain) is believed to be stabilized by four cysteines engaged in two disulfide bonds. These four cysteines as well as their relative positions are well conserved in all species. The strict evolutionary conservation of this domain and its similarity to cystatins, a family of proteinase inhibitors, suggests it plays specific and independent biologic function in host defense.

The C-terminal 37 amino acids (LL-37) of the mature AMP of human cationic antibacterial protein of 18 kDa (hCAP18) has been characterized. LL-37 was originally referred to as FALL39, named for the first 4 N-terminal amino acids (phe-ala-leu-leu) of this domain and the total number of residues (i.e., 39). LL-37 is a peptide predicted to contain an amphipathic alpha helix and lacks cysteine, making it different from all other previously isolated human peptide antibiotics of the defensin family, each of which contain 3 disulfide bridges. Antibacterial peptides from different mammals contained a conserved pro-region very similar to cathelin. Full length hCAP18 comprises the cathelin-like precursor protein and the C-terminal LL-37 peptide, thus comprising 170 amino acids. It is contemplated to generate a fusion polypeptide comprising a cathelicidin domain or a cathelin-like domain operably linked to a delta haemolysin or phenol soluble modulin-delta or function variant thereof.

Generally, the antibiotic or other antimicrobial is administered in a bactericidal, antiviral and/or antifungal amount. The peptide provides for a method of increasing antibiotic activity by permeabilizing the bacterial outer membrane and combinations involving peptide and a sub-inhibitory amount (e.g., an amount lower than the bactericidal amount) of antibiotic can be administered. Typically, the delta haemolysin or phenol soluble modulin-delta or functional variant and antibiotic are administered within 48 hours of each other (e.g., 2-8 hours, or may be administered simultaneously). A “bactericidal amount” is an amount sufficient to achieve a bacteria-killing blood concentration in the subject receiving the treatment. In accordance with its conventional definition, an “antibiotic,” as used herein, is a chemical substance that, in dilute solutions, inhibits the growth of, or kills microorganisms. Also encompassed by this term are synthetic antibiotics (e.g., analogs) known in the art.

The polypeptides or peptides of the disclosure can be used, for example, as preservatives or sterillants of materials susceptible to microbial or viral contamination. For example, the peptides can be used as preservatives in processed foods (e.g., to inhibit organisms such as Salmonella, Yersinia, and Shigella). If desired, the peptides can be used in combination with antibacterial food additives, such as lysozymes. The peptides of the disclosure can also be used as a topical agent, for example, to inhibit Pseudomonas or Streptococcus or kill odor-producing microbes (e.g., Micrococci). The optimal amount of a delta haemolysin or phenol soluble modulin-delta or functional variant of the disclosure for any given application can be readily determined by one of skill in the art.

Intracellular mature virions (IMV) of vaccinia have a double layer membrane of endoplasmic reticulum derived membrane cisternae. As the IMV migrates through an infected cell the virion acquires a double layer outer envelope consisting of a cellular cisternae known as a wrapping membrane and become intracellular enveloped virions. Egress from the cell is accompanied by fusion of the outermost layer with the plasma membrane yielding a three layer outer membrane on extra-cellular enveloped virions (EEV). Both the IMV and EEV forms are infectious with the EEV being most efficient in cell entry. Delta haemolysin or phenol soluble modulin-delta or a functional variant, and related homologues are effective at disrupting the IMV and EEV of the virions thus being useful as antiviral agents.

The disclosure includes the use of delta haemolysin or phenol soluble modulin-delta functional variants for treatment of viral skin disease, especially for the treatment of vaccinia and small pox infection. As the molecules are proteins, they are most well suited for topical application. However, peptidomimetics and other protein analogs with more favorable pharmacokinetic and pharmacodynamic properties can be developed for use with other routes of administration including, but not limited to, oral and parenteral. The polypeptides can be incorporated into appropriate delivery devices dependent upon the route of administration and other considerations well known to those skilled in the art. Additionally, as delta haemolysin or phenol soluble modulin-delta or functional variant are peptides, the coding sequence could be delivered to the site of interest using any gene transfer protocol to allow for expression of the gene product.

The delta haemolysin or phenol soluble modulin-delta or functional variants can be used in conjunction with vaccination to ameliorate or prevent eczema vaccination or after vaccination for the treatment of skin conditions.

The disclosure provides delta haemolysin or phenol soluble modulin-delta and functional variants which have antiviral activity. The delta haemolysin or phenol soluble modulin-delta and functional variants are useful for inhibiting viral infection or spread, as well reducing the effects of viral infection. One or more delta haemolysin or phenol soluble modulin-delta or functional variant peptides can be used, for example, as an antiviral agent in topical lotions as well as in other pharmaceuticals including soaps and wipes. A delta haemolysin or phenol soluble modulin-delta or functional variant of the disclosure can be used alone or in combination with conventional antiviral agents and can be used as an adjunct therapy.

The effect of delta haemolysin or phenol soluble modulin-delta and functional variants on vaccinia virus have not been previously reported. The mechanism of action for these peptides is hypothesized to involve disruption of the microbial membrane and/or the penetration of the microbial membranes to interfere with intracellular functions.

Eczema Vaccinatum (EV) is one of the major complications of small pox vaccination and occurs in patients with a history of atopic dermatitis (AD), a Th2-mediated skin disease. Recently it was found that AD skin is deficient in its ability to express certain endogenous antimicrobial peptides. This group of patients is known to be much more susceptible to serious complications of infection with vaccinia and related viruses. Vaccinia virus is used for small pox vaccination.

The disclosure also provides a method for inhibiting the spread or infection of a virus by contacting the virus or a surface upon which a virus may be present with an inhibiting effective amount of a delta haemolysin or phenol soluble modulin-delta or functional variant peptide of the disclosure. The term “contacting” refers to exposing the virus to a cationic antiviral peptide so that the peptide can inhibit the spread of infectivity of a virus or kill the virus itself. For example, by adding a delta haemolysin or phenol soluble modulin-delta functional variant to a culture comprising a virus (e.g., vaccinia virus) one can measure the susceptibility of a culture to the infectivity of a virus in the presence and absence of a delta haemolysin or phenol soluble modulin-delta functional variant. Alternatively, contacting can occur in vivo, for example, by administering a delta haemolysin or phenol soluble modulin-delta or functional variant to a subject that is susceptible to or afflicted with a viral infection. The administration includes topical as well as parenteral. “Inhibiting” or “inhibiting effective amount” refers to the amount of a peptide that is sufficient to cause a viral inhibition or kill a virus. Examples of viruses that can be inhibited include herpesviridae (herpes simplex virus (HSV), varicella-zoster virus), vaccinia virus, Pappiloma virus and other viruses causing skin diseases. The method for inhibiting the viral infection can also include the contacting of a virus with a delta haemolysin or phenol soluble modulin-delta or functional variant alone or in combination with one or more other antiviral agents.

The delta haemolysin or phenol soluble modulin-delta functional variants are also useful as a broad-spectrum antimicrobials suitable for tackling the growing problem of antibiotic-resistant bacteria strains, and for treating and/or preventing outbreaks of infectious diseases, including diseases caused by bioterrorism agents like anthrax, plague, cholera, gastroenteritis, multidrug-resistant tuberculosis (MDR TB). The delta haemolysin or phenol soluble modulin-delta or functional variants and kits of the disclosure can be used therapeutically and prophylactically for biodefense against new bioattacks. For example, the disclosure provides kits containing formulations comprising a delta haemolysin or phenol soluble modulin-delta or functional variant of the disclosure alone or in combination with cathlicidins or other antimicrobial agents. The kits can be provided, for example, to a population subject to bioterrorist attacks (e.g., the military).

The disclosure provides a method for inhibiting viral infection and spread of such viruses as herpesviridae (herpes simplex virus (HSV), varicella-zoster virus), vaccinia virus, Pappiloma virus and other viruses causing skin diseases, as well as diseases and disorders associated with atopic dermatitis by administering a therapeutically effective amount of a delta haemolysin or phenol soluble modulin-delta or functional variant of the disclosure to a subject who has, or is at risk of having, such an infection or disorder. The term “inhibiting” means preventing or ameliorating infectivity of a virus or a sign or symptoms of a disorder (e.g., atopic dermatitis). Examples of disease signs that can be ameliorated include skin sores and lesions associated with herpesviridae (herpes simplex virus (HSV), varicella-zoster virus), vaccinia virus, Pappiloma virus and other viruses causing skin infection such as those seen in atopic dermatitis. Examples of patients who can be treated in the disclosure include those at risk for, or those suffering from, a viral infection, such as those resulting from Herpesviridae (herpes simplex virus (HSV), varicella-zoster virus), vaccinia virus, Pappiloma virus and other viruses causing skin diseases. Those skilled in the art of medicine can readily employ conventional criteria to identify appropriate subjects for treatment in accordance with the disclosure.

The delta haemolysin or phenol soluble modulin-delta or functional variant of the disclosure can be used, for example, as preservatives or sterillants of materials susceptible to viral contamination. For example, the peptides can be used as preservatives in processed foods, as spray disinfectants commonly used in the household or clinical environment. The optimal amount of a cationic peptide of the disclosure for any given application can be readily determined by one of skill in the art.

In another aspect, the disclosure provides knockout non-human animals that are useful to screen potential antiviral and antibacterial delta haemolysin or phenol soluble modulin-delta functional variants and agents useful for treating such diseases and disorders as atopic dermatitis.

As a model to study the potential in vivo significance of a delta haemolysin or phenol soluble modulin-delta, CRAMP Cnlp knockout mice known to lack expression of CRAMP, a close murine ortholog of cathelicidin human LL-37, can be used. Importantly these mice generated a significantly greater number of pox skin lesions than seen in wild type isogenic control mice. Accordingly, one can screen the biological activity of a variant of delta haemolysin or phenol soluble modulin-delta using the CRAMP knockout susceptible mice. These in vitro and in vivo observations suggest that the increased susceptibility of atopic dermatitis patients to eczema vaccinatum may be due to a deficiency of cathelicidin. Such knockout mice are effective models to test the therapeutic effects of delta haemolysin or phenol soluble modulin-delta functional variants.

In one aspect, the disclosure provides a method of screening for biologically active antimicrobial agents comprising a delta haemolysin or phenol soluble modulin-delta or functional variant comprising contacting a culture comprising a Staphylococcus or group A Streptococcus with a delta haemolysin or phenol soluble modulin-delta or functional variant and determine the effect the agent has on bacterial growth or the effect on bacterial killing. In another aspect, an in vivo model can be used comprising generating an infection on a CRAMP knockout mouse and detecting the ability of a delta haemolysin or phenol soluble modulin-delta or functional variant thereof to reduce the infection or symptoms of the infection following contacting the infected mouse with a delta haemolysin or phenol soluble modulin-delta or functional variant thereof.

The following EXAMPLES are provided to further illustrate but not limit the invention.

### EXAMPLES

### Example 1

In vitro studies showed that S. epidermidis inhibits both GAS and S. aureus. When S. epidermidis is plated atop GAS, an inhibition zone extends beyond the S. epidermidis colony, which suggests the production of a soluble antimicrobial compound (FIG. 6a). To ascertain whether the antimicrobial is secreted, rather than membrane bound, GAS growth was monitored in cell-free S. epidermidis, Propionibacterium acnes, and GAS supernatants. GAS growth was entirely inhibited only in S. epidermidis supernatants, denying the suspicion of a membrane and further suggesting the production and secretion of an antimicrobial (FIG. 6b).

Several strains of S. epidermidis, including pathogenic catheter isolates 1457 and RP62A and non-pathogenic isolate ATCC 12228, were assayed for activity and it was found that all strains produced compounds toxic to GAS. Using a radial diffusion assay and GAS survival kinetics, activity was observed in the pathogenic strains after partial purification and concentration (FIG. 6c,d). Similarly, it was found that antimicrobial compounds from S. epidermidis inhibited and killed S. aureus (FIG. 7).

S. epidermidis also inhibited growth of GAS on human and mouse skin. The presence of S. epidermidis on human skin prevented the survival of GAS and subsequent hemolysis when printed on blood agar (FIG. 6e). On freshly excised mouse skin, the presence of both S. epidermidis strains greatly reduced and in some cases eliminated GAS survival (FIG. 6f). These data suggest that the presence of S. epidermidis on epithelial surfaces protects the skin from GAS infections by production of a soluble, secreted compound.

### Example 2

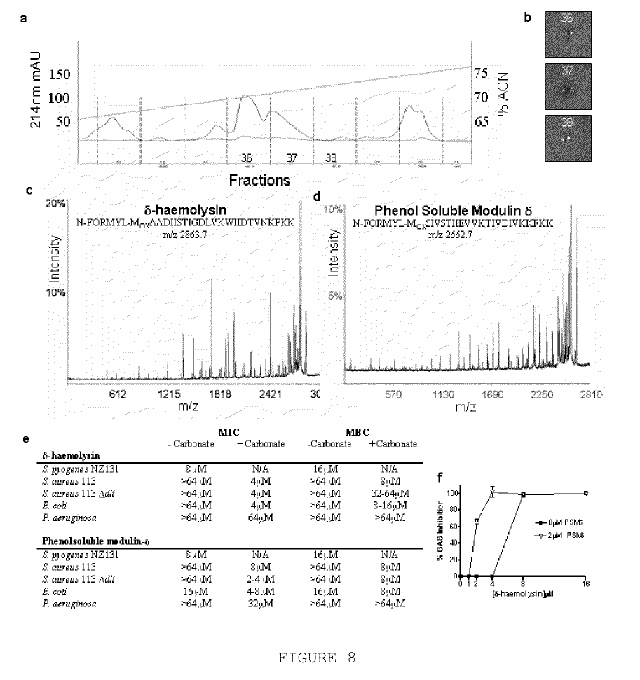

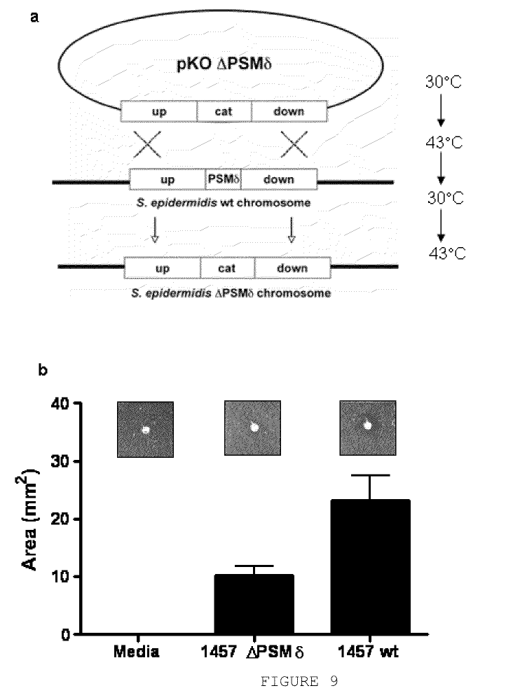

In order to determine the compounds responsible for S. epidermidis inhibition of GAS, stationary phase S. epidermidis supernatant was purified by a reversed-phase C18 column and HPLC fractionation (FIG. 8a,b). The antimicrobial fraction 37, determined through radial diffusion assay, was sequenced by tandem MALDI TOF mass spectrometry (FIG. 8b). Sequencing and blast results yielded 2 peptide sequences: delta-haemolysin and Phenol Soluble Modulin-delta (PSMdelta) (FIG. 8c,d). In order to determine if these compounds could be responsible for the antimicrobial activity and protection against GAS, synthetic versions of these peptides were assayed against a variety of microorganisms in the presence and absence of carbonate (FIG. 8e). Both synthetic peptides showed significant inhibitory and bactericidal activity against GAS, S. aureus, E. coli, and to a lesser degree, P. aeruginosa. Since the two peptides eluted simultaneously, whether the peptides interact to form a potent antimicrobial complex was investigated through co-incubation with GAS. The MIC of delta-haemolysin against GAS was found to be 8 μM. In the presence of 2 μM PSMdelta, the MIC of delta-haemolysin against GAS was reduced to 4 μM (FIG. 8f).

To illustrate the importance of PSMdelta in vivo, an allelic replacement mutant in S. epidermidis 1457 was created. The psmdelta gene was replaced with a gene encoding chloramphenicol acetyltransferase (cat), the peptide conferred resistance to chloramphenicol (FIG. 9a). This psmdelta knockout (deltapsmdelta) was constructed using a temperature sensitive plasmid, containing cat flanked by regions of the S. epidermidis genome homologous to regions directly surrounding psmdelta. Through a series of temperature shifts, the gene was removed and replaced by cat. PCR confirmed the presence of the cat in place of psmdelta in the chromosome. Internal gene primers also confirmed the absence of psmdelta in the chromosome of S. epidermidis. To determine if PSMdelta was partially responsible for the antimicrobial activity of S. epidermidis against GAS, first partially purified the supernatants of S. epidermidis WT and S. epidermidis deltapsmdelta was obtained. Radial diffusion assay showed that S. epidermidis WT supernatants produced a zone of inhibition of 23.2 mm², 44% greater than the zone produced by S. epidermidis deltapsmdelta, 10.3 mm² (FIG. 9b). These results indicate that PSMdelta plays an important role in GAS inhibition. The remaining antimicrobial activity found in S. epidermidis deltapsmdelta supernatants likely occurs from the other antimicrobial peptide, delta-haemolysin.

### Example 3

Previous reports of S. epidermidis delta-haemolysin suggest that the peptide plays a role in quorum sensing and bacterial growth regulation at the genetic level. In addition, other studies have reported that delta-hemolysin from S. aureus, a peptide similar to that produced by S. epidermidis, interacts with lipid membranes of the bacterial cell wall. To assess how delta-haemolysin and PSMdelta function as antimicrobial peptides, their physical properties were investigated.

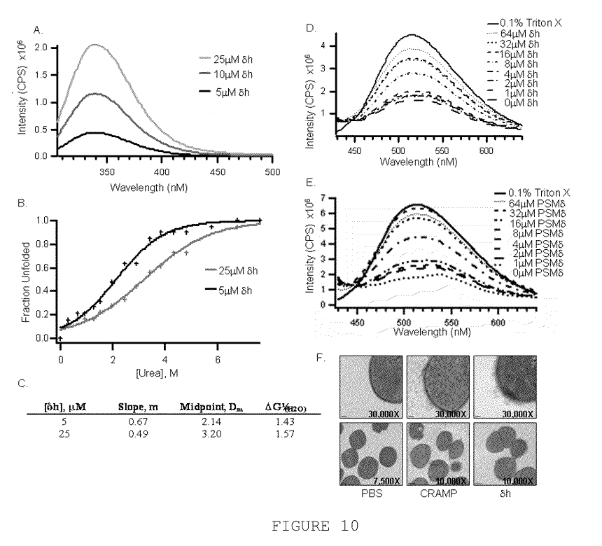

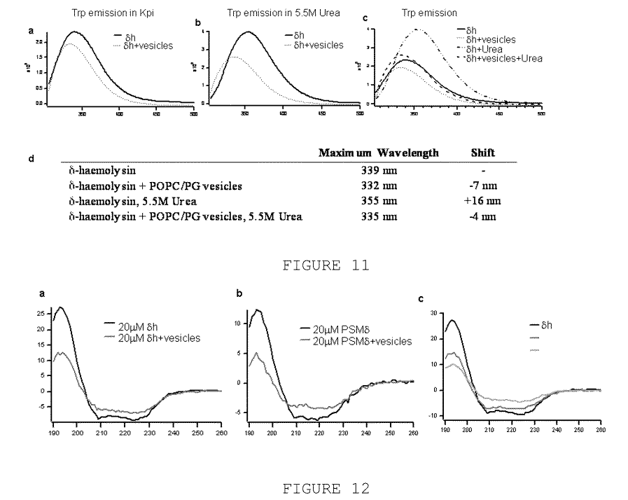

Tryptophan emission is a sensitive indicator for the physical environment surrounding the amino acid (˜330 nm in a folded or hydrophobic environment; ˜355 nm in an unfolded or aqueous environment). The tryptophan in delta-haemolysin showed only partial exposure to the aqueous environment in buffer alone, suggesting that the peptides aggregate. The peptides were dissociated or unfolded from their complexes using increasing concentrations of urea (FIG. 10a,b). Increasing peptide concentration (5 to 25 μM) shifted the unfolding curve such that higher concentrations of urea were required to disassemble the peptide complexes (FIG. 10b). The midpoints of the unfolding curves of 5 μM and 25 μM were 2.14 and 3.20M urea and yielded deltaG¹(H₂O) values of 1.43 and 1.57 kcal/mol, respectively (FIG. 10c). These data indicate that increasing the concentration (number of peptides) increases the deltaG¹(H₂O) and thus, the stability of multimeric peptide complexes. Furthermore, upon delta-haemolysin incubation with lipid vesicles, the tryptophan emission blue shifts from 339 nm to 332 nm, suggesting that the peptides associate with the membrane (FIG. 11a, d). Interestingly, urea is unable to dissociate the peptide from the vesicle, as the tryptophan only slightly red shifts from 332 nm to 335 nm (FIG. 11b,d). Thus, delta-haemolysin forms multimeric peptide complexes and strongly interacts with membranes.

Both delta-haemolysin and PSMdelta were evaluated for their ability to perforate synthetic lipid vesicles. Lipid vesicles were made with a 2:1 molar ratio of POPC to POPG. The lipid vesicles, extruded through a 200 nm polycarbonate film, encapsulated the fluorescent dye, ANTS, and a quencher: DPX. Upon membrane perforation, ANTS/DPX are released and separated, allowing ANTS to fluoresce at 530 nm. Dose-dependent fluorescence was observed when delta-haemolysin and PSMdelta were incubated with lipid vesicles for 1 hour (FIG. 10d, e). In addition, SEM analysis of GAS treated with delta-haemolysin showed membrane blebbing and disruption, similar to GAS treated with CRAMP. These data indicate that the peptides function similar to innate antimicrobials of the skin, disrupting and lysing target membranes.

These observations show that S. epidermidis competes with and prevents growth of skin pathogens. The data also show that the peptides can cause membrane leakage and are toxic. Despite this relative toxicity, S. epidermidis harmlessly and ubiquitously resides on the skin's surface and is unable to invade tissue unabated, which suggests that the bacterium has a non-pathogenic tendency. In addition, the layer of cornified epithelium likely renders the skin impenetrable to the antimicrobial peptides produced by S. epidermidis, making the peptides a defense against pathogenic invaders such as GAS and S. aureus.

While S. epidermidis protects the skin from infections, the skin affords the bacterium an ecological niche conducive to growth and survival. Healthy skin, unlike burned-skin, supports survival and growth of the bacterium, illustrating that S. epidermidis benefits directly from the cutaneous niche. The reciprocated benefit derived from the colonization of S. epidermidis on the skin classifies this bacterium as a mutual symbiote, rather than a commensal. Thus, S. epidermidis and other cutaneous microbiota play a vital role in directly promoting host health, and indirectly influencing the epidermal cells. The positive benefits of these microbes and their products indicate not only their inclusion in the host innate immune system, but also their position as the first line of defense against invading pathogens.

### Example 4

Bacteria were grown in Tryptic soy broth (TSB) for 15-16 hours. Supernatants were used for preparation of 10 KDa filtrates. Neonatal human epidermal keratinocytes and mouse macrophages were used for in vitro experiments for S. epidermidis 10 KDa extract or Staphylococcal LTA stimulations.

Cells were stimulated for 24 hours with the indicated stimuli. RNA was isolated for Real-time RT-PCR. Concentration of TNFα in cell culture supernatant was measured by enzyme-linked immunosorbent assay (ELISA) according to manufacture's instruction.

Quantitative Real-time RT-PCR was performed in an ABI PRISM 7000 sequence detector (PE Applied Biosystems). The primers and probes used for TNFα, IL-6, IL-8, INFα, INFβ, MIP2, TRAF1, A20, IRAK-M, hBD2, hBD3, cathelicidin and GAPDH were purchased from Applied Biosystems.

Immunofluorescent staining and western blot assay were used to detect TRAF1 induced by S. epidermidis 10 KDa extract and Staphylococcal LTA. S. epidermidis-derived LTA antibody was used for blocking the activity of Staphylococcal LTA.

100 nM caspase-8 inhibitor was added into cells 10 minutes before poly(I:C) or/and S. epidermidis 10 KDa extracts, LTA-SA were added. After 48-hour incubation, the supernatants from cell culture were collected for TNFα ELISA.

Four pairs of siRNA oligonucleotides targeted to TRAF1, TLR2 (Dharmacon; SMART Pool) and non-targeted control siRNA (Dharmacon) were used. The efficiency of blockage in these experiments was 55% for TRAF1, 34% for TLR2 by real time RT-PCR. After 24 or 48 hours transfection, poly(I:C) or/and S. epidermidis 10 KDa extracts, LTA-SA were added to stimulate cells for 24 hours. The production of TNFα was evaluated by ELISA.

C57BL/6 wild-type and TLR2-deficient mice were used for in vivo experiments. 12 μg S. epidermidis 10 KDa extract or 40 μg LTA-SA was injected in mouse ear lobes 1-2 hour before 100 μg poly(I:C) was injected. After 24 hours, ears were cut and homogenized by bead beater (BIOSPEC PRODUCTS). RNA was isolated from ears by using Trizol Reagent. The expressions of TRAF1 and cytokines were analyzed by real-time RT-PCR.

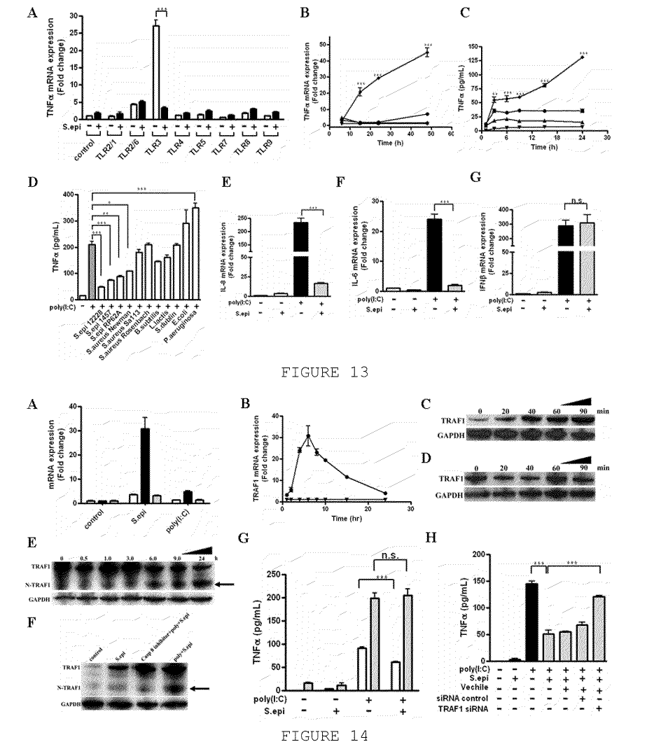

Polyriboinosinic polyribocytidylic acid [poly(I:C)] continuously induced the production of TNFα in normal human keratinocytes (FIGS. 13b and 13c). And this induction was suppressed by S. epidermidis 10 KDa filtrate (FIG. 13a) in time-dependent manner on mRNA and protein levels (FIGS. 13b and 13c). To test whether the suppression of TLR3-dependent TNFα is specific by S. epidermidis 10 KDa filtrate, the different fractions from S. epidermidis and heat-killed S. epidermidis were compared. TLR3-dependent TNFα was dramatically suppressed by S. epidermidis 10 KDa filtrate and less-efficiently suppressed by S. epidermidis whole extract, but not by other fractions or heat-killed bacterium. The lactate dehydrogenase (LDH) cytotoxicity assay showed the suppression by the 10 KDa filtrate was not due to the cytotoxicity of the fraction to result in the lethality of normal human keratinocytes. Different 10 KDa filtrates from eight Gram-positive and three Gram-negative bacteria were also examined. KDa filtrates from three S. epidermidis strains and one Staphylococcus aureus strain (Staphylococcus aureus Newman) significantly suppressed TLR3-dependent TNFα (FIG. 13d), but not the 10 KDa filtrates from other Gram-positive bacteria (S. aureus Sa113, S. aureus Rosenbach, Bacillus subtilis and Lactococcus lactis) and three Gram-negative strains (Escherichia coli ATCC29522, Salmonella dublin Lane, Pseudomonas aeruginosa). Notably, 10 KDa filtrates from S. epidermidis strains were high-efficient suppressors of TLR3-dependent TNFα than that from S. aureus Newman (FIG. 13d). To rule out the possibility that S. epidermidis 10 KDa filtrate was restricted to suppress TLR3-dependent TNFα, another proinflammatory cytokines and some antimicrobial peptides were tested. S. epidermidis 10 KDa filtrate also suppressed the production of TLR3-dependent IL-8 and IL-6 (FIGS. 13e and 13f), but had no effect on INFα and INFβ (FIG. 13g). Remarkably, S. epidermidis 10 KDa filtrate synergistically induced the expression of antimicrobial peptides hBD2 and hBD3 with poly(I:C), whereas poly(I:C) suppressed the expression of cathelicidin, which was induced by S. epidermidis 10 KDa filtrate. The immunofluorescent staining showed the filtrate prevented the activation of transcription factor NF-κB via the blockade of NF-κB translocation into nucleus (FIG. 13h). Taken together, the results demonstrate that S. epidermidis 10 KDa filtrate play an important role in suppressing the production of proinflammatory cytokines without decreasing innate immunity and indicate that S. epidermidis 10 KDa filtrate controls the upstream of TLR3 signaling pathway to eliminate these cytokines.

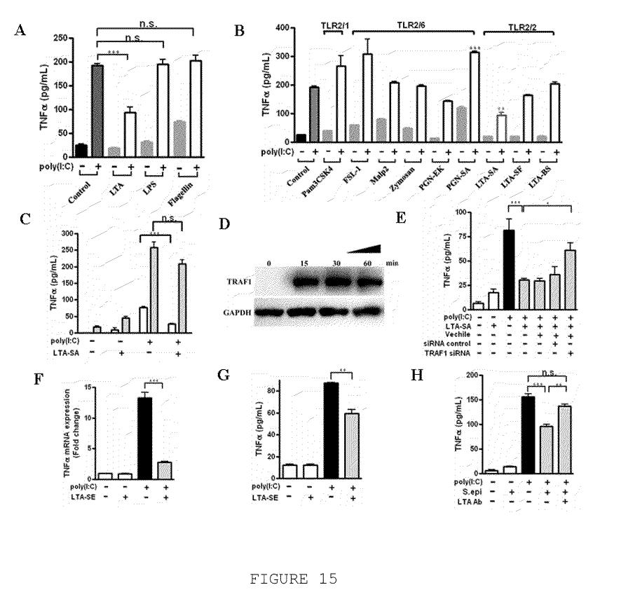

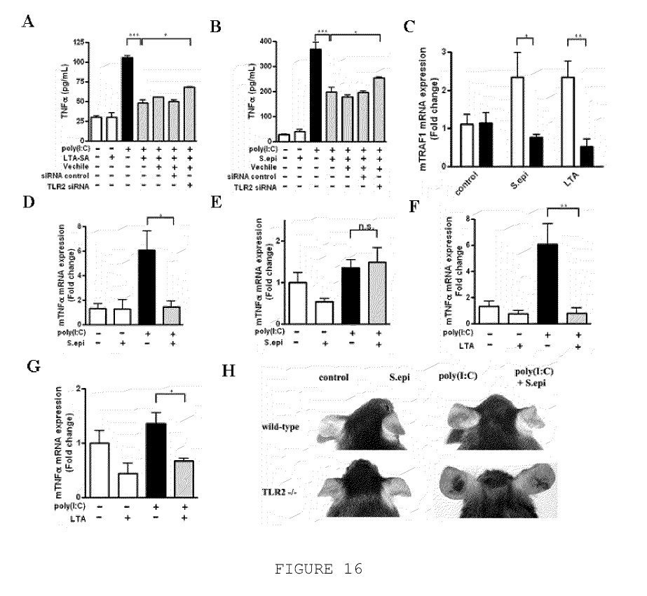

Three negative regulators, TRAF1, A20 and IRAK-M were examined. In normal human keratinocytes, TRAF1, but not A20 and IRAK-M, was induced by S. epidermidis 10 KDa filtrate (FIG. 14a). None of three negative regulators was induced by poly(I:C) (FIGS. 14a and 14d). The induction of TRAF1 by S. epidermidis was in time-dependent manner on mRNA and protein levels by RT-PCR and western blot analyses (FIGS. 14b and 14c). Immunofluorescent staining showed TRAF1 induced by S. epidermidis 10 KDa filtrate located in the cytoplasm in normal human keratinocytes. To serve as a negative regulator of TLR3 signaling, TRAF1 needs to be cleaved by caspase 8 to release N-terminal TRAF1 (N-TRAF1), which binds to TRIF for terminating TLR3 signaling. In normal human keratinocytes the inductions of TRIF and caspase 8 by poly(I:C) were observed. The overexpression of TRIF caused caspase 8-dependent cleavage of TRAF1 to release N-terminal TRAF1 (N-TRAF1) (FIG. 14e), which was prevented by caspase 8 inhibitor (FIG. 14f). To determine whether caspase 8 is responsible for the activation of functional TRAF1 to suppress TLR3-dependent TNFα, caspase 8 inhibitor was pre-incubated with normal human keratinocytes before poly(I:C) and S. epidermidis 10 KDa filtrate was added to induce functional TRAF1. The suppression of TLR3-dependent TNFα by S. epidermidis 10 KDa was completely abrogated by caspase 8 inhibitor, suggesting caspase 8 is required for releasing functional TRAF1 to turn off TLR3 signaling. To define S. epidermidis-induced TRAF1 is a regulator involved in turning off TLR3 signaling to produce TNFα, RNA interference was used to knock down TRAF1 in normal human keratinocytes. After TRAF1 was knocked down, 83% of TNFα suppressed by S. epidermidis 10 KDa filtrate was restored (FIG. 14h). These findings demonstrate that poly(I:C)-activated caspase 8 plays an essential role in the modification of S. epidermidis-induced TRAF1 to release functional part of TRAF1 (N-TRAF1), and N-TRAF1 serve as a negative regulator of TLR3 signaling to limit the production of TNFα.

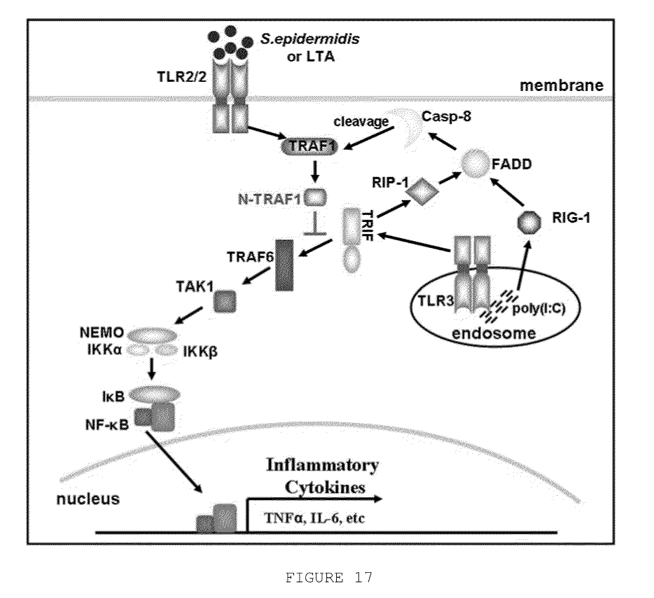

As a Gram-positive bacterium, LTA and phenol-soluble modulin from S. epidermidis can trigger innate immunity through TLR2 signaling. To explore the molecule from S. epidermidis which serves as suppressor of TLR3-dependent TNFα, different TLR2 ligands were combined with poly(I:C). Combinations of TLR2/1, TLR2/6 ligands with poly(I:C) synergistically induced TNFα (S. aureus-derived PGN) or failed to suppress TNFα (FIG. 15a). Surprisingly, only LTA from S. aureus (LTA-SA) significantly suppress the production of TLR3-dependent TNFα, but not LTAs from Bacullus subtilis and Streptococcus faecium (FIG. 15a). And the suppression by LTA-SA was abolished by caspase 8 inhibitor (FIG. 15b). Western blot analysis and immunofluorescent staining showed LTA-SA also induced TRAF1 (FIG. 15c), suggesting LTA-SA might function as S. epidermidis 10 KDa filtrate to induce TRAF1 for the suppression of TLR3-dependent TNFα. To confirm this hypothesis, RNA interference of TRAF1 was used. After TRAF1 was knocked down, the production of TLR3-dependent TNFα was partially restored (FIG. 15d), supporting TRAF1 is the regulator induced by LTA-SA to suppress TNFα and indicating LTA might be the molecule from S. epidermidis to suppress TLR3-dependent TNFα. To define LTA is the molecule from S. epidermidis serving as the suppressor of TLR3-dependent TNFα, LTA was extracted from S. epidermidis by butonal and crude LTA from S. epidermidis (LTA-SE) did suppress the production of TLR3-dependent TNFα in normal human keratinocytes (FIG. 15e). Furthermore, the specific antibody that reacts with Staphylococcal LTA can prevent it from suppressing the production of TLR3-dependent TNFα. Thus, the findings demonstrate that S. epidermidis regulate TRAF1 through TLR2 signaling to terminate TLR3 signaling to produce TNFα.

Given the importance of macrophages in innate immunity and inflammation the studies were extended to mouse macrophage. Real-time RT-PCR analysis showed elevated expression of TNFα in mouse macrophage when the cells had been challenged with poly(I:C) and S. epidermidis 10 KDa filtrate or Staphylococcal LTA. Furthermore, S. epidermidis 10 KDa filtrate or LTAs alone induced TNFα expression in mouse macrophage, which was not found in human kerationcytes (FIG. 13a, FIGS. 15b and 15e), but consistent with the previous reports that staphylococci and Staphylococcal LTA induced the production of TNFα in mouse macrophage and human monocytes. Moreover, the combination of poly(I:C) and TLR2 agonists synergistically up-regulated the production of TNFα in a primary human PBMC culture system. Therefore, these studies implicate that the suppression of TLR3-dependent TNFα by S. epidermidis 10 KDa filtrate or Staphylococcal LTA might be restricted in skin keratinocyte.

To dissect the mechanism responsible for S. epidermidis to regulate TRAF1, the production of TLR3-dependent TNFα was measured after TLR2 was knocked down in normal human keratinocytes. 33% and 35% of TLR3-dependent TNFα suppressed by S. epidermidis 10 KDa filtrate and Staphylococcal LTA were restored, respectively (FIGS. 16a and 16b), suggesting S. epidermidis 10 KDa filtrate or Staphylococcal LTA limits TLR3-dependent TNFα through TLR2 signaling. To identify that S. epidermidis 10 KDa filtrate and Staphylococcal LTA regulate TRAF1 through TLR2 signaling TLR2-deficient mice were used. Expression of TRAF1 induced by S. epidermidis 10 KDa filtrate and LTA-SA was evaluated in C57BL/6 wild-type and TLR2^{-/-} mice. Compared to wild-type mice, S. epidermidis 10 KDa filtrate or Staphylococcal LTA failed to induce TRAF1 in TLR2^{-/-} mice (FIG. 16c). Therefore, the expression of TRAF1 observed in wild-type mice was induced through TLR2 signaling by S. epidermidis 10 KDa filtrate or Staphylococcal LTA. To determine whether TLR2 is responsible for S. epidermidis or Staphylococcal LTA to regulate TRAF1 to suppress TLR3-dependent TNFα in vivo, the ear lobes of C57BL/6 TLR2^{+/+} and TLR2^{-/-} mice were challenged intradermally with poly(I:C), S. epidermidis 10 KDa filtrate and Staphylococcal LTA. Consequently, TLR3-dependent TNFα was not suppressed by S. epidermidis 10 KDa in TLR2^{-/-} mice compared to that in wild-type mice (FIGS. 16d and 16e). Furthermore, the expression of TLR3-dependent IL-6 and MIP2 was also suppressed by S. epidermidis 10 KDa filtrate as well as TNFα in both C57BL/6 and BABL/c wild-type mice, but not in C57BL/6 TLR2^{-/-} mice. Surprisingly, although LTA-SA completely suppressed TLR3-dependent TNFα and IL-6 in wild-type mice (FIG. 16f), it maintained its ability to partially suppress the expression of these two cytokines (26% for TNFα and 88% for IL-6) in TLR2^{-/-} mice (FIG. 16g). Interestingly, poly(I:C) was less-efficient to induce TNFα in TLR2^{-/-} mice (FIGS. 16e and 16g), indicating that TLR2 might be involved in TLR3 signaling to produce TNFα.

These experiments reveal an important new role of skin inhabitant microbe S. epidermidis: suppressing excessive the production of proinflammatory cytokines caused by viral infections. S. epidermidis triggers TLR2 signaling to induce TRAF1, which was cleaved by caspase 8 that was recruited and activated by poly(I:C) through TLR3 signaling. The cleaved N-TRAF1 serves as a suppressor for TLR3 signaling to eliminate the production of proinflammatory cytokines (FIG. 17). Whereas less-efficient induction of TNFα by poly(I:C) and the retained ability of Staphylococcal LTA to suppress TLR3-dependent TNFα in TLR2^{-/-} mice suggest there might be another molecule or signaling pathway involved in limiting TLR3-dependent proinflammatory cytokines. Regardless, TRAF1 plays a pivotal role in the elimination of TLR3-dependent TNFα, thereby making it a potentially important target for future therapeutic strategies.

A number of embodiments of the disclosure have been described. Nevertheless, it will be understood that various modifications may be made without departing from the spirit and scope of the disclosure. Accordingly, other embodiments are within the scope of the following claims.

1. A method for inhibiting the growth of a bacterium or yeast comprising contacting the bacterium or yeast with an inhibiting effective amount of a composition comprising a delta-haemolysin and/or a phenol soluble modulin-delta.

2. (canceled)

3. (canceled)

4. The method of claim 1, further comprising contacting the bacterium or yeast with at least one additional antimicrobial agent.

5. The method of claim 4, wherein the antimicrobial agent is selected from the group consisting of a cathelicidin peptide, β-lactam, novobiocin, polymyxin B, and LL-37.

6. The method of claim 1, wherein the contacting is in vitro.

7. The method of claim 1, wherein the contacting is in vivo.

8. The method of claim 7, wherein the contacting is by topical administration.

9. The method of claim 1, wherein the delta-haemolysin comprises an amino acid sequence as set forth in SEQ ID NO:2, 3, 4, 5, 6 or variants thereof having from 1 to 10 conservative amino acid substitutions.

10. The method of claim 1, wherein the phenol soluble modulin-delta comprises an amino acid sequence as set forth in SEQ ID NO:7 of a variant thereof having from 1 to 10 conservative amino acid substitutions.

11. The method of claim 1, wherein the delta-haemolysin and/or phenol soluble modulin-delta composition comprises a pharmaceutically acceptable carrier.

12. (canceled)

13. (canceled)

14. The method of claim 9, wherein the delta-haemolysin comprising SEQ ID NO:2, 3, 4, 5, or 6 comprises a D-amino acid.

15. The method of claim 10, wherein the phenol soluble modulin-delta comprising SEQ ID NO:7 comprises a D-amino acid.

16. The method of claim 1, wherein the composition comprises a recombinant host cell that expresses a delta haemolysin or phenol soluble modulin-delta or functional variant thereof.

17. A method of treating infections or dermatological disorders comprising administering an effective amount of Staphylococcus epidermidis (S. epidermidis), or an effective amount of an extract of S. epidermidis comprising the peptide delta-haemolysin and/or phenol soluble modulin-delta.

18. The method of claim 17, wherein the infection comprises a bacterial, fungal, parasitic or viral infection.

19. The method of claim 17, wherein the dermatological disorders comprise wounds, diabetic ulcers, acne, rosacea, atopic dermatitis, pyodermas, burn wounds, catheter infections, Group A Streptococcus infections, Staphylococcus aureus infections or other dermatological diseases.

20. (canceled)

21. (canceled)

22. The method of claim 17 in combination therapy with antimicrobial agents to treat dermatological problems such as wounds, diabetic ulcers, acne, rosacea, atopic dermatitis, pyodermas, burn wounds, catheter infections, Group A Streptococcus infections, Staphylococcus aureus infections or other dermatological diseases.

23. The method of claim 17, wherein the treatment is for wounds, diabetic ulcers, acne, rosacea, atopic dermatitis, pyodermas, burn wounds, catheter infections, Group A Streptococcus infections, Staphylococcus aureus infections or other dermatological diseases in immunocompromised individuals.

24. The method of claim 17, wherein the treatment is for cancer.

25. A composition comprising (i) S. epidermidis, (ii) a recombinant host cell that expresses a polypeptide comprising delta-haemolysin or phenol soluble modulin-delta or functional variant thereof; (iii) an extract of (i) or (ii) comprising a delta haemolysin or phenol soluble modulin-delta or functional variant thereof; and (iv) delta haemolysin or phenol soluble modulin-delta or a functional variant thereof.

26. The composition of claim 25, further comprising a cathelicidin or other antimicrobial peptide.

27. (canceled)

28. (canceled)

29. A composition comprising an LTA agent or a 10 KDa fraction from S. epidermidis or S. aureus in a pharmaceutically acceptable carrier.

30. A method of treating a TNFα-associated inflammatory disease or disorder comprising contacting a subject with a composition of claim 29.

31. The method of claim 30, wherein the disease or disorder is a dermal or epidermal disease or disorder.

32. (canceled)

33. (canceled)
